SEQUENCE LISTING (7 sequences)

SEQ ID NO: 1 - DNA, 2264 bp, Artificial Sequence
  Synthetic
atggaggctgtggctgtggctgccgctgtgggagtgctgctgctggctggggccggaggggccgctggcgatgaggctcgggaggctgcagctgtgcgcgcactggtcgcacgactgctgggacctgggccagcagccgacttctctgtgagtgtcgagagagccctggctgcaaagcccggactggatacctacagtctgggaggaggaggagcagctcgagtgagggtcagagggtcaacaggagtggcagcagctgcaggactgcaccgatatctgcgagacttttgcggctgtcatgtggcctggtcaggaagccagctgaggctgcccagacctctgccagcagtgccaggcgagctgacagaagccactcccaaccggtaccgctactatcagaacgtgtgcacccagtcctattctttcgtctggtgggactgggctcgatgggagcgcgaaatcgattggatggcactgaacggaattaatctggcactggcatggtccggacaggaggcaatctggcagagagtgtacctggcactgggactgactcaggccgagattaacgagttcttcaccgggccagcttttctggcatggggacggatggggaatctgcacacatgggacggaccactgccaccttcttggcacatcaaacagctgtatctgcagcatagggtgctggatcagatgagaagttttggcatgactccagtgctgcccgctttcgcaggacacgtccctgaggccgtgacacgcgtcttcccacaggtgaacgtcactaagatgggcagctggggacacttcaactgcagttactcatgtagcttcctgctggcccctgaagatccaatttttcccatcattggaagcctgttcctgcgggagctgatcaaagaatttggaaccgaccacatctacggggccgatacattcaacgagatgcagccacccagctccgaacctagctacctggccgctgcaaccacagccgtgtacgaggccatgaccgctgtggacacagaagccgtctggctgctgcaggggtggctgtttcagcatcagccacagttctggggacctgcacagatccgagctgtgctgggagcagtcccacgaggaaggctgctggtgctggatctgttcgctgagtcccagcccgtctacactaggaccgcctctttccagggccagccttttatttggtgtatgctgcacaactttggagggaatcatgggctgttcggcgcactggaggcagtgaacggaggaccagaagcagctagactgtttcctaatagcactatggtgggcaccggaatggctcccgagggcatctcacagaatgaagtggtctacagcctgatggcagagctgggatggcgaaaggaccccgtgcctgatctggcagcctgggtcactagtttcgctgcaaggagatacggggtgtcacaccctgacgctggagcagcttggcgactgctgctgaggagcgtgtacaactgcagtggggaggcctgtagaggccataatcggtccccactggtgcggcgcccctcactgcagatgaacaccagcatctggtacaatcgatccgacgtgttcgaagcttggcggctgctgctgacaagtgccccttcactggctacttctccagcattccgctatgacctgctggatctgacaaggcaggccgtgcaggagctggtcagcctgtactatgaggaagctcgcagcgcatacctgtccaaggaactggcatccctgctgagggcaggaggcgtgctggcttatgagctgctgccagctctggacgaagtcctggcatccgattctagatttctgctgggcagctggctggagcaggcacgagcagcagccgtgagcgaggccgaagctgacttctacgagcagaactctaggtatcagctgactctgtggggacccgaagggaacatcctggattacgcaaacaagcagctggcaggactggtggctaattactatacccctagatggcggctgtttctggaggccctggtggactctgtcgctcagggcattccattccagcagcaccagtttgataagaacgtgttccagctggaacaggccttcgtcctgtctaaacagcggtatcctagtcagccacgcggcgacacagtggacctggcaaagaagattttcctgaaatactatccccgctgggtggctggctcatggaccggtggcgcgccttcgaactgcagctcgaggta

SEQ ID NO: 2 - DNA, 4533 bp, Artificial Sequence
  Synthetic
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctcagatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgaagatctaggcctgcaggcggccgccaccatggaggctgtggctgtggctgccgctgtgggagtgctgctgctggctggggccggaggggccgctggcgatgaggctcgggaggctgcagctgtgcgcgcactggtcgcacgactgctgggacctgggccagcagccgacttctctgtgagtgtcgagagagccctggctgcaaagcccggactggatacctacagtctgggaggaggaggagcagctcgagtgagggtcagagggtcaacaggagtggcagcagctgcaggactgcaccgatatctgcgagacttttgcggctgtcatgtggcctggtcaggaagccagctgaggctgcccagacctctgccagcagtgccaggcgagctgacagaagccactcccaaccggtaccgctactatcagaacgtgtgcacccagtcctattctttcgtctggtgggactgggctcgatgggagcgcgaaatcgattggatggcactgaacggaattaatctggcactggcatggtccggacaggaggcaatctggcagagagtgtacctggcactgggactgactcaggccgagattaacgagttcttcaccgggccagcttttctggcatggggacggatggggaatctgcacacatgggacggaccactgccaccttcttggcacatcaaacagctgtatctgcagcatagggtgctggatcagatgagaagttttggcatgactccagtgctgcccgctttcgcaggacacgtccctgaggccgtgacacgcgtcttcccacaggtgaacgtcactaagatgggcagctggggacacttcaactgcagttactcatgtagcttcctgctggcccctgaagatccaatttttcccatcattggaagcctgttcctgcgggagctgatcaaagaatttggaaccgaccacatctacggggccgatacattcaacgagatgcagccacccagctccgaacctagctacctggccgctgcaaccacagccgtgtacgaggccatgaccgctgtggacacagaagccgtctggctgctgcaggggtggctgtttcagcatcagccacagttctggggacctgcacagatccgagctgtgctgggagcagtcccacgaggaaggctgctggtgctggatctgttcgctgagtcccagcccgtctacactaggaccgcctctttccagggccagccttttatttggtgtatgctgcacaactttggagggaatcatgggctgttcggcgcactggaggcagtgaacggaggaccagaagcagctagactgtttcctaatagcactatggtgggcaccggaatggctcccgagggcatctcacagaatgaagtggtctacagcctgatggcagagctgggatggcgaaaggaccccgtgcctgatctggcagcctgggtcactagtttcgctgcaaggagatacggggtgtcacaccctgacgctggagcagcttggcgactgctgctgaggagcgtgtacaactgcagtggggaggcctgtagaggccataatcggtccccactggtgcggcgcccctcactgcagatgaacaccagcatctggtacaatcgatccgacgtgttcgaagcttggcggctgctgctgacaagtgccccttcactggctacttctccagcattccgctatgacctgctggatctgacaaggcaggccgtgcaggagctggtcagcctgtactatgaggaagctcgcagcgcatacctgtccaaggaactggcatccctgctgagggcaggaggcgtgctggcttatgagctgctgccagctctggacgaagtcctggcatccgattctagatttctgctgggcagctggctggagcaggcacgagcagcagccgtgagcgaggccgaagctgacttctacgagcagaactctaggtatcagctgactctgtggggacccgaagggaacatcctggattacgcaaacaagcagctggcaggactggtggctaattactatacccctagatggcggctgtttctggaggccctggtggactctgtcgctcagggcattccattccagcagcaccagtttgataagaacgtgttccagctggaacaggccttcgtcctgtctaaacagcggtatcctagtcagccacgcggcgacacagtggacctggcaaagaagattttcctgaaatactatccccgctgggtggctggctcatggtaagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 3 - protein, 738 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYFLSRTQTTGGTANTQTLGFSQGGPNTMANQAKNWLPGPCYRQQRVSTVTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRFLTRNL

SEQ ID NO: 4 - DNA, 145 bp, adeno-associated virus 2
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 5 - DNA, 6838 bp, Artificial Sequence
  Synthetic
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattcatggaggctgtggctgtggctgccgctgtgggagtgctgctgctggctggggccggaggggccgctggcgatgaggctcgggaggctgcagctgtgcgcgcactggtcgcacgactgctgggacctgggccagcagccgacttctctgtgagtgtcgagagagccctggctgcaaagcccggactggatacctacagtctgggaggaggaggagcagctcgagtgagggtcagagggtcaacaggagtggcagcagctgcaggactgcaccgatatctgcgagacttttgcggctgtcatgtggcctggtcaggaagccagctgaggctgcccagacctctgccagcagtgccaggcgagctgacagaagccactcccaaccggtaccgctactatcagaacgtgtgcacccagtcctattctttcgtctggtgggactgggctcgatgggagcgcgaaatcgattggatggcactgaacggaattaatctggcactggcatggtccggacaggaggcaatctggcagagagtgtacctggcactgggactgactcaggccgagattaacgagttcttcaccgggccagcttttctggcatggggacggatggggaatctgcacacatgggacggaccactgccaccttcttggcacatcaaacagctgtatctgcagcatagggtgctggatcagatgagaagttttggcatgactccagtgctgcccgctttcgcaggacacgtccctgaggccgtgacacgcgtcttcccacaggtgaacgtcactaagatgggcagctggggacacttcaactgcagttactcatgtagcttcctgctggcccctgaagatccaatttttcccatcattggaagcctgttcctgcgggagctgatcaaagaatttggaaccgaccacatctacggggccgatacattcaacgagatgcagccacccagctccgaacctagctacctggccgctgcaaccacagccgtgtacgaggccatgaccgctgtggacacagaagccgtctggctgctgcaggggtggctgtttcagcatcagccacagttctggggacctgcacagatccgagctgtgctgggagcagtcccacgaggaaggctgctggtgctggatctgttcgctgagtcccagcccgtctacactaggaccgcctctttccagggccagccttttatttggtgtatgctgcacaactttggagggaatcatgggctgttcggcgcactggaggcagtgaacggaggaccagaagcagctagactgtttcctaatagcactatggtgggcaccggaatggctcccgagggcatctcacagaatgaagtggtctacagcctgatggcagagctgggatggcgaaaggaccccgtgcctgatctggcagcctgggtcactagtttcgctgcaaggagatacggggtgtcacaccctgacgctggagcagcttggcgactgctgctgaggagcgtgtacaactgcagtggggaggcctgtagaggccataatcggtccccactggtgcggcgcccctcactgcagatgaacaccagcatctggtacaatcgatccgacgtgttcgaagcttggcggctgctgctgacaagtgccccttcactggctacttctccagcattccgctatgacctgctggatctgacaaggcaggccgtgcaggagctggtcagcctgtactatgaggaagctcgcagcgcatacctgtccaaggaactggcatccctgctgagggcaggaggcgtgctggcttatgagctgctgccagctctggacgaagtcctggcatccgattctagatttctgctgggcagctggctggagcaggcacgagcagcagccgtgagcgaggccgaagctgacttctacgagcagaactctaggtatcagctgactctgtggggacccgaagggaacatcctggattacgcaaacaagcagctggcaggactggtggctaattactatacccctagatggcggctgtttctggaggccctggtggactctgtcgctcagggcattccattccagcagcaccagtttgataagaacgtgttccagctggaacaggccttcgtcctgtctaaacagcggtatcctagtcagccacgcggcgacacagtggacctggcaaagaagattttcctgaaatactatccccgctgggtggctggctcatggaccggtggcgcgccttcgaactgcagctcgaggtaggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccagctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgggctgca

SEQ ID NO: 6 - protein, 739 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQFFLYYLSRTQTTGGTANTQTLGFSQGGPNTMANQAKNWLPGPCYRQQRVSTVTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRFLTRNL

SEQ ID NO: 7 - DNA, 2232 bp, Artificial Sequence
  Synthetic
atggaggctgtggctgtggctgccgctgtgggagtgctgctgctggctggggccggaggggccgctggcgatgaggctcgggaggctgcagctgtgcgcgcactggtcgcacgactgctgggacctgggccagcagccgacttctctgtgagtgtcgagagagccctggctgcaaagcccggactggatacctacagtctgggaggaggaggagcagctcgagtgagggtcagagggtcaacaggagtggcagcagctgcaggactgcaccgatatctgcgagacttttgcggctgtcatgtggcctggtcaggaagccagctgaggctgcccagacctctgccagcagtgccaggcgagctgacagaagccactcccaaccggtaccgctactatcagaacgtgtgcacccagtcctattctttcgtctggtgggactgggctcgatgggagcgcgaaatcgattggatggcactgaacggaattaatctggcactggcatggtccggacaggaggcaatctggcagagagtgtacctggcactgggactgactcaggccgagattaacgagttcttcaccgggccagcttttctggcatggggacggatggggaatctgcacacatgggacggaccactgccaccttcttggcacatcaaacagctgtatctgcagcatagggtgctggatcagatgagaagttttggcatgactccagtgctgcccgctttcgcaggacacgtccctgaggccgtgacacgcgtcttcccacaggtgaacgtcactaagatgggcagctggggacacttcaactgcagttactcatgtagcttcctgctggcccctgaagatccaatttttcccatcattggaagcctgttcctgcgggagctgatcaaagaatttggaaccgaccacatctacggggccgatacattcaacgagatgcagccacccagctccgaacctagctacctggccgctgcaaccacagccgtgtacgaggccatgaccgctgtggacacagaagccgtctggctgctgcaggggtggctgtttcagcatcagccacagttctggggacctgcacagatccgagctgtgctgggagcagtcccacgaggaaggctgctggtgctggatctgttcgctgagtcccagcccgtctacactaggaccgcctctttccagggccagccttttatttggtgtatgctgcacaactttggagggaatcatgggctgttcggcgcactggaggcagtgaacggaggaccagaagcagctagactgtttcctaatagcactatggtgggcaccggaatggctcccgagggcatctcacagaatgaagtggtctacagcctgatggcagagctgggatggcgaaaggaccccgtgcctgatctggcagcctgggtcactagtttcgctgcaaggagatacggggtgtcacaccctgacgctggagcagcttggcgactgctgctgaggagcgtgtacaactgcagtggggaggcctgtagaggccataatcggtccccactggtgcggcgcccctcactgcagatgaacaccagcatctggtacaatcgatccgacgtgttcgaagcttggcggctgctgctgacaagtgccccttcactggctacttctccagcattccgctatgacctgctggatctgacaaggcaggccgtgcaggagctggtcagcctgtactatgaggaagctcgcagcgcatacctgtccaaggaactggcatccctgctgagggcaggaggcgtgctggcttatgagctgctgccagctctggacgaagtcctggcatccgattctagatttctgctgggcagctggctggagcaggcacgagcagcagccgtgagcgaggccgaagctgacttctacgagcagaactctaggtatcagctgactctgtggggacccgaagggaacatcctggattacgcaaacaagcagctggcaggactggtggctaattactatacccctagatggcggctgtttctggaggccctggtggactctgtcgctcagggcattccattccagcagcaccagtttgataagaacgtgttccagctggaacaggccttcgtcctgtctaaacagcggtatcctagtcagccacgcggcgacacagtggacctggcaaagaagattttcctgaaatactatccccgctgggtggctggctcatggtaa

(19) United States
(12) Patent Application Publication
(10) Pub. No.: US 2023/0183741 A1
(43) Pub. Date: Jun. 15, 2023

(54) DISEASE CORRECTION BY DELIVERY OF AAV8 VECTORS EXPRESSING CODON OPTIMIZED NAGLU

(71) Applicant: University of Florida Research Foundation, Incorporated, Gainesville, FL (US)

(72) Inventors: Coy D. Heldermon, Gainesville, FL (US); Sanford L. Boye, Gainesville, FL (US)

(73) Assignee: University of Florida Research Foundation, Incorporated, Gainesville, FL (US)

(21) Appl. No.: 17/924,539

(22) PCT Filed: May 11, 2021
(86) PCT No.: PCT/US2021/031863

§ 371 Date: Nov. 10, 2022

Related U.S. Application Data
(60) Provisional application No. 63/023,224, filed on May 11, 2020.

(51) Int. Cl. C12N 15/86; C12N 9/24

(52) U.S. Cl. CPC C12N 15/86; C12N 9/2402; C12Y 302/0105; C12N 2750/14122; C12N 2750/14143; C12N 2750/14145; C12N 2800/22

(57) ABSTRACT

The present disclosure provides AAV8 vectors and variants thereof that express nucleic acids sharing identity to a codon optimized NAGLU (coNAGLU) that improves transduction and distribution in brain cells and will improve disease outcomes in the Mucopolysaccharidoses IIIB (MPS IIIB) mouse model. The present disclosure also provides methods of treatment of a subject, and methods of transducing one or more brain cells, by administering these vectors, as well as uses of these vectors in the manufacture of medicaments for treatment. The present disclosure also provides compositions and host cells comprising rAAV vectors and rAAV particles that express a coNAGLU heterologous nucleic acid and confer enhanced transduction efficiency in human cells, such as brain cells (e.g., neurons). These compositions may be administered to a subject in need thereof.

### RELATED APPLICATIONS

This application claims the benefit of the filing date of U.S. Provisional Application No. 63/023,224, filed May 11, 2020, the entire contents of which are incorporated by reference.

### GOVERNMENT SUPPORT

This invention was made with government support under Grant Number R01 NS102624 awarded by the National Institutes of Health. The Government has certain rights in the invention.

### BACKGROUND OF THE INVENTION

Mucopolysaccharidoses IIIB (MPS IIIB) is an autosomal recessive lysosomal storage disease caused by mutations in N-acetylglucosaminidase (NAGLU). MPS IIIB results in aberrant retention of heparan sulfate within lysosomes and progressive central nervous system (CNS) degeneration. MPS IIIB is also known as Sanfilippo Syndrome, type B. Clinical signs include hyperactivity, loss of social interaction and progressive mental degeneration, which eventually results in severe impairment of neurocognitive ability and loss of motor function Death in human MPS IIIB patients often occurs between the ages of 15 and 20 years. The well-characterized MPS IIIB mouse model has been utilized to test therapeutic interventions and possesses many of the same biochemical, histological and clinical features as the human disease. There is currently no cure for MPS IIIB.

Gene therapy has great potential for treating lysosomal storage diseases; however, widespread gene delivery throughout the central nervous system (CNS) remains a major challenge due to the need to overcome the blood brain barrier. The recombinant adeno-associated virus (AAV) vector system has been a favored gene delivery tool and has proven highly efficient in transducing post-mitotic cells in a wide range of tissues, including the CNS. Further, no significant pathogenesis has been linked to AAV in humans. Direct administration of rAAV viral vectors into brain parenchyma can serve to bypass the blood brain barrier, thereby allowing targeted transduction of neuronal cells. Indeed, successful AAV-mediated gene therapy has been demonstrated in MPS models, which resulted in improvements in neuropathology, behavior and increased lifespan. However, differential responses are observed based on the serotype of AAV that is administered and age at vector administration. Several studies suggest that the greatest therapeutic advantage would stem from early treatment intervention, before disease pathology becomes evident and irreversible.

Improvement of MPS IIIB symptoms following intraparenchymal injection in vivo of AAV5 vectors expressing NAGLU has been demonstrated. See Heldermon et al., Gene Ther. 2013 September; 20(9): 913-921, herein incorporated by reference. And it was previously demonstrated that a six site intracranial administration of AAV2/5 NAGLU resulted in improvement in brain heparan sulfate storage, hearing, motor coordination and lifespan but did not completely correct the disease. See C. D. Heldermon, et al., Mol. Ther. 18 (2010) 873-880, herein incorporated by reference. Thus, there remains a need in the art for novel gene therapies, such as novel AAV vectors and capsids, that confer substantially enhanced transduction in brain cells and thus provide partial or complete correction of Sanfilippo syndrome symptoms and increase patient survival.

### SUMMARY OF THE INVENTION

The present disclosure provides recombinant AAV (rAAV) vectors of serotype AAV8 and variants thereof that express nucleic acids sharing identity to a codon optimized NAGLU (coNAGLU) that improves transduction in brain cells and improves disease outcomes in the Mucopolysaccharidoses IIIB (MPS IIIB) mouse model. In exemplary embodiments, the coNAGLU has been codon-optimized for human expression. In some embodiments, the coNAGLU is a full-length human NAGLU. The rAAV vectors of the disclosure may be packaged into viral particles. The present disclosure also provides methods of treatment of a subject, and methods of transducing one or more brain cells, by administering these vectors and particles, as well as uses of these vectors in the manufacture of medicaments for treatment. In particular embodiments, the nucleic acid coding sequences (or heterologous nucleic acid sequence) of the rAAV vectors shares at least 85% identity to a coNAGLU. (In the literature, “NAGLU” may refer to N-acetylglucosaminidase or alpha-N-acetylglucosaminidase, which are the same enzyme.) Codon-optimized NAGLU may also be referred to herein as “codon-modified” or “cm”.

The present disclosure provides compositions and host cells comprising rAAV vectors and rAAV particles that express a coNAGLU heterologous nucleic acid and confer enhanced transduction efficiency in human cells, such as brain cells (e.g., neurons). These compositions may be administered to a subject in need thereof, such as a human subject.

This disclosure is based, at least in part, on the recent demonstration that AAV8 provides a broader transduction area and higher transduction efficiency in the MPS IIIB mouse brain compared to AAV5, AAV9, and AAVrh10. See Gilkes et al., Gene Ther. 2016; 23: 263-271 and Gilkes, Bloom & Heldermon, Molecular Genetics and Metabolism Reports, 6 (2016) 48-54, both of which are herein incorporated by reference. The NAGLU protein is about 745 amino acids in length, and the NAGLU coding sequence is about 2230 nucleotides in length.

Accordingly, in some aspects, the disclosure provides a nucleic acid comprising NAGLU that has been codon optimized for expression in human cells and is adapted for use in AAV nucleic acid vectors. The disclosure also provides improved recombinant AAV8 vectors, and variants thereof, for expression of coNAGLU in human brain cells. The disclosure also provides the use of AAV8 capsid variants comprising mutations to amino acid residues associated with ubiquitination of the AAV vector in expression of coNAGLU. In particular embodiments, the disclosure provides a triple-capsid mutant (tcm) modification to the AAV8 capsid designed to reduce ubiquitination and degradation of the AAV vector, which confers further enhanced expression and distribution in brain cells. In some aspects, the brain cells comprise cells of the cerebral cortex, hippocampus, thalamus or cerebellum. In exemplary embodiments, the brain cells comprise cerebral cortex cells.

In some aspects, the disclosed rAAV vectors comprise inverted terminal repeats (ITRs) from an AAV of serotype 2 (AAV2). In some aspects, the coNAGLU of the disclosed vectors is operably controlled by a promoter, such as a chicken beta actin (CBA) promoter.

In some aspects, the recombinant AAV (rAAV) vectors of the disclosure may be used to treat a subject who has or is at risk of having, or is diagnosed with Sanfilippo Syndrome B or MPS IIIB. Accordingly, provided herein are methods of treatment of Sanfilippo Syndrome B. The subject may be a mammal, such as a human. In some embodiments, any of the disclosed methods results in partial or complete restoration of hearing loss in the subject, or partial or complete restoration of normal (or baseline) levels of heparan sulfate storage in the subject. In some embodiments, the methods provide for partial or complete restoration of normal neurological (e.g., auditory) function.

In some aspects, the disclosure provides rAAV particles comprising any of the disclosed AAV vectors, and pharmaceutical compositions comprising any of the disclosed vectors and one or more pharmaceutically acceptable excipients. In some embodiments of the disclosed methods, any of the disclosed rAAV particles or pharmaceutical compositions is administered by intraparenchymal injection (e.g., intraparenchymal six site (IC6) injection) or cisternal injection. In some embodiments, administration of any of the disclosed rAAV particles or pharmaceutical compositions is by intracerebroventricular (ICV) injection, intrathalamic injection, or ventral tegmental area (VTA) injection. In particular embodiments, administration is by intraparenchymal six site (IC6) injection.

In some aspects, the disclosure provides host cells comprising any of the disclosed rAAV vectors. The host cell may be a mammalian cell, such as a human cell. The host cell may be a neuron or glia, such as a human neuron or glia.

The Examples disclosed herein provide the results of a study using intracranial six site (IC6) injections and cisternal injection of AAVtcm8-coNAGLU vector compared to untreated controls to determine disease correction as estimated by the effects on enzyme activity, CNS immune activation, coordination, activity level, hearing, and survival. Histology and enzymatic assays show that both injection methods result in above normal levels of NAGLU expression in the brain.

In some aspects, the heterologous nucleic acid sequence encoding a NAGLU may be codon-optimized for expression in mammals other than humans, such as rodents (e.g., Mus musculus). In some aspects, the heterologous nucleic acid sequence is not a wild-type NAGLU (cDNA) sequence.

### BRIEF DESCRIPTION OF THE DRAWINGS

The following drawings form part of the present specification and are included to demonstrate certain aspects of the present invention. The invention may be better understood by reference to the following description taken in conjunction with the accompanying drawings, in which like reference numerals identify like elements, and in which:

FIGS. 1A-1B depict running wheel activity data, in which male mice (N=8-10 per group) were exposed to 12-hour light/dark cycles, and their running activity was recorded using the Clocklab software. FIG. 1A shows the proportion of total activity performed while ambient lights were on. This data showed a trend toward more daytime activity in untreated MPS IIIB mice, with improvements in both treated groups. FIG. 1B depicts activity onset time in 4-5 month old mice, while ambient lights were off. * denotes p<0.05, and ** denotes p<0.01. Abbreviations: “no tx” denotes an untreated group; IC6=intracranial injection; ICM=intracisternal magna injection.

FIG. 2 show antibody staining of Lysosomal-associated membrane protein 1 (LAMP) in the cerebral cortex. Brains from 6 month old mice for each group were fixed and immunohistological analyses for LAMP1 were performed. AAVTM=AAVtcm8-coNAGLU vector administration

FIGS. 3A-3D depict NAGLU enzymatic activity in the four mice groups, as measured in four different sections of the central nervous system: the forebrain, hindbrain, cerebellum, and spinal cord. Enhancement of NAGLU enzymatic activity is shown as an X-fold change relative to the normal untreated group (“Con”).

FIG. 4 depicts auditory brainstem response (ABR) thresholds measurements in the four groups of mice. ABR thresholds were measured using tone burst stimuli at 8, 16, and 32 kHz in mice at 9 months of age. Two-way ANOVA with Bonferroni's multiple comparisons tests were used. Data are shown as means±SEM. *p<0.05, Norm untreated vs. Mut untreated, **p<0.05, Mut untreated vs. Mut IC6, ***p<0.05, Mut untreated vs. Mut ICM.

FIG. 5 depicts survival proportions data in each of the four groups of mice.

FIG. 6 shows antibody staining of Lysosomal-associated membrane protein 1 (LAMP) in the cerebellum.

FIG. 7 shows auditory-evoked brainstem response (ABR) of the four groups of mice at 8 and 16 kHz.

FIG. 8 is a schematic overview of an exemplary regimen of treatment and response assessment for administration via the IC6 and ICM routes of tcmAAV8-NAGLU into MPSIIIB mice.

FIG. 9 depicts an assessment of transduction efficiency of AAV8, AAV8 (double Y-F), and AAV8 (double Y-F+T-V) when injected via the IC6 method. Three-month-old MPS IIIB mice were assessed for GFP expression and tissue penetration into the cortex, hippocampus, thalamus, and cerebellum. Low magnification images of the entire mid-sagittal section are shown below regional high magnification images. Images obtained using ScanScope FL. Cortex, thalamus, and cerebellum (×20), scale bar=100 μm; hippocampus (×8), scale bar=300 μm.

FIG. 10 shows that use of AAV8 (double Y-F+T-V) results in superior GFP intensity levels. Brains of 3-month-old MPS IIIB animals injected with AAV8, AAV8 (double Y-F), or AAV8 (double Y-F+T-V) via the IC6 method were collected and processed. Cortical, hippocampal, thalamic, and cerebellar limits of sagittal tissue sections were deli-neated and differences in mean GFP intensity levels across the regions for each animal and vector injected were assessed using the Image Studio Lite software and quantitated using one-way ANOVA. Data were normalized to AAV8 (double Y-F) GFP intensity. *p<0.05; **p<0.01; ^{$} p<0.0001; n=4.

FIG. 11 shows that the route of AAV administration impacts tissue penetration and biodistribution. AAV8 (double Y-F+T-V) was injected into neonatal MPS IIIB animals via the IC6, ICV, TH, and VTA methods. Brains were extracted at 3 months, sectioned into four structurally unique, relatively equidistant sections, in millimeters from midline −4.2 (section 1), −3.72 (section 2), −2.72 (section 3), and −1.72 (section 4), respectively, and assessed for differences in tissue penetration and GFP biodistribution as a consequence of route of administration. Images obtained using ScanScope FL. Scale bar=2 mm.

FIG. 12 shows that the IC6 method of administration results in the most widespread GFP biodistribution within the brain. Using 3-month-old MPS IIIB animals, differences in GFP biodistribution as a con-sequence of method of administration were quantitatively assessed in each of the four tissue sections; as well as, cumulatively as indicated in the figure legend. Data were analyzed by two-way ANOVA. Data represented as mean±SEM. *p<0.05; **p<0.01; ^{#}p<0.001; ^{$}p<0.0001; n=5 for IC6 and n=3 each for ICV, TH, and VTA.

FIG. 13 shows that CNS administration of AAV8 (double Y-F+T-V) results in somatic transduction of organs. Three months after IC6 and ICV routes of AAV administration, somatic organs were harvested and assessed for presence of GFP. Both injection methods result in preferential transduction of heart and liver, indicating vector entry to systemic circulation. ×20 images obtained with ScanScope FL. Scale bar=300 μm.

### DETAILED DESCRIPTION OF THE INVENTION

The present disclosure is related to improved compositions and methods related to gene therapy treatments. Specifically, provided herein are therapeutic rAAV vectors and methods related to treatment of Sanfilippo Syndrome, e.g., Sanfilippo Syndrome type B. The disclosed rAAV vectors may be used to treat other symptoms of diseases and conditions associated with NAGLU, such as Charcot-Marie-Tooth disease type 2V. The present disclosure provides compositions comprising rAAV vectors and rAAV particles that express a N-acetylglucosaminidase alpha (NAGLU) gene, and in particular a codon-optimized NAGLU (coNAGLU) gene that may be administered to a subject in need thereof. Administration of any of the disclosed compositions of rAAV vectors, e.g., in accordance with the treatment methods described herein, may also confer enhanced distribution of NAGLU protein in brain cells, such as in lysosomes. In particular, administration of these compositions may confer improved lysosomal integrity in the brain cells of a subject. These compositions may confer improved storage and retention of heparan sulfate in cells of the subject. Further, administration of these compositions may confer partial or complete restoration of hearing loss in the subject.

The present disclosure further provides methods of transducing expression of NAGLU in one or more brain cells comprising administering to the cells any of the disclosed rAAV vectors. These methods may confer enhanced biodistribution of the NAGLU protein in the cells as well as enhanced transduction. The present disclosure also provides host cells comprising these rAAV-coNAGLU vectors and particles and compositions comprising these rAAV-coNAGLU vectors.

Further provided herein are methods for preventing Sanfilippo syndrome type B (or MPS IIIB) or inhibiting progression of the disease in a mammal (e.g., a human), the method comprising administering to the mammal any one of the disclosed rAAV vectors, as well as uses of these vectors as medicaments. Also provided herein are methods for preventing other types of Sanfilippo syndrome, such as types A, C, and D.

In some aspects, the disclosure provides a composition for use in treating Sanfilippo syndrome, and a composition for use in the manufacture of a medicament to treat Sanfilippo syndrome. In some aspects, the disclosure provides a composition comprising an rAAV particle as described herein for use in treatment by administering to the brain of a mammal (e.g., a human), for example, by intraparenchymal injection. In some embodiments, methods of administration of the disclosed particles and vectors do not comprise administrations to the retina or the liver of a subject.

The rAAV vectors of the disclosure may be of serotype AAV8, or a variant thereof. AAV8-based vectors have been shown to have better transduction efficiency and distribution in brain cells than AAV vectors of other serotypes. See Gilkes et al., Gene Ther. 2016; 23: 263-271 and Gilkes, Bloom & Heldermon, Molecular Genetics and Metabolism Reports, 6 (2016) 48-54. In some embodiments, the disclosed rAAV vectors are encapsidated in an AAV8 capsid. In some embodiments, the disclosed rAAV vectors are encapsidated in a capsid that is not of serotype 8. In some embodiments, the disclosed rAAV vectors are encapsidated in an AAV8 capsid variant. In some embodiments, the rAAV vectors are pseudotyped, e.g., are of an rAAV2/8 pseudotype. Accordingly, the disclosure provides rAAV particles of serotype AAV8, or a variant thereof. The disclosure provides further rAAV particles of an AAV2/8 pseudotype. In other embodiments, the disclosure provides rAAV particles of an AAV9 capsid serotype.

In particular embodiments, the capsids of the disclosed rAAV vectors and particles have one or more non-native amino acid substitutions relative to the wild-type AAV8 capsid protein sequence. In some embodiments, the capsid comprises non-native amino acid substitutions at one or more of the amino acid residues 447, 494, and 733 of a wild-type AAV8 capsid. In some embodiments, the capsid comprises substitutions of phenylalanine (F) at one or more of residues tyrosine 447 and tyrosine 733 of a wild-type AAV8 capsid. In some embodiments, the capsid comprises substitution of a valine at residue threonine 494 of a wild-type AAV8 capsid. In some embodiments, the capsid comprises one or more of the following mutations in the wild-type AAV8 capsid sequence: Y444F, Y447F, Y733F, and T494V. In particular embodiments, the capsid is an AAV8(Y447F+Y733F+T494V) capsid [a triple mutant], comprising each of the three amino acid substitutions. The AAV8(Y447F+Y733F+T494V) capsid is described in Kay et al., Targeting Photoreceptors via Intravitreal Delivery Using Novel, Capsid-Mutated AAV Vectors, PLoS One. 2013, 8(4), e6209, herein incorporated by reference. In some embodiments, the capsid comprises an amino acid sequence that has at least 95% identity, at least 98% identity, or at least 99% identity to the AAV8(Y447F+Y733F+T494V) VP1 capsid protein sequence of SEQ ID NO: 3. In some embodiments, the capsid comprises the amino acid sequence of SEQ ID NO: 3, which is provided below.

(SEQ ID NO: 3)
MAADGYLPDWLEDNLSEGIREWWALKPGAPKPKAN
QQKQDDGRGLVLPGYKYLGPFNGLDKGEPVNAADA
AALEHDKAYDQQLQAGDNPYLRYNHADAEFQERLQ
EDTSFGGNLGRAVFQAKKRVLEPLGLVEEGAKTAP
GKKRPVEPSPQRSPDSSTGIGKKGQQPARKRLNFG
QTGDSESVPDPQPLGEPPAAPSGVGPNTMAAGGGA
PMADNNEGADGVGSSSGNWHCDSTWLGDRVITTST
RTWALPTYNNHLYKQISNGTSGGATNDNTYFGYST
PWGYFDFNRFHCHFSPRDWQRLINNNWGFRPKRLS
FKLFNIQVKEVTQNEGTKTIANNLTSTIQVFTDSE
YQLPYVLGSAHQGCLPPFPADVFMIPQYGYLTLNN
GSQAVGRSSFYCLEYFPSQMLRTGNNFQFTYTFED
VPFHSSYAHSQSLDRLMNPLIDQYLYFLSRTQTTG
GTANTQTLGFSQGGPNTMANQAKNWLPGPCYRQQR
VSTVTGQNNNSNFAWTAGTKYHLNGRNSLANPGIA
MATHKDDEERFFPSNGILIFGKQNAARDNADYSDV
MLTSEEEIKTTNPVATEEYGIVADNLQQQNTAPQI
GTVNSQGALPGMVWQNRDVYLQGPIWAKIPHTDGN
FHPSPLMGGFGLKHPPPQILIKNTPVPADPPTTFN
QSKLNSFITQYSTGQVSVEIEWELQKENSKRWNPE
IQYTSNYYKSTSVDFAVNTEGVYSEPRPIGTRFLT
RNL

The capsid of the rAAV particle may be an AAV8(Y447F+Y733F+T494V) or an AAV8(Y447F+Y733F) capsid variant. In some embodiments, the capsid of the rAAV particle is an AAV8(Y447F) capsid variant. In some embodiments, the capsid is an AAV8(T494V) capsid variant. In some embodiments, the capsid is an AAV8(Y447F+Y733F) [a double mutant] capsid variant. In other embodiments, the capsid of the disclosed rAAV particles comprises a non-native amino acid substitution at amino acid residues 533 and/or 733 of a wild-type AAV8 capsid, wherein the non-native amino acid substitution is E533K, Y733F, or a combination thereof. The AAV8(Y733F) capsid is described in Doroudchi et al., Amer. Soc. of Gene & Cell Ther. 19(7): 1220-29 (2011), herein incorporated by reference. In some embodiments, the modified capsid comprises AAV7BP2, another variant of AAV8.

In some aspects, the capsid of the rAAV particle is an AAV8(Y444F+Y733F+T494V) or the AAV8(Y444F+Y733F) capsid variant. In exemplary embodiments, the capsid of the rAAV particle is an AAV8(Y444F+Y733F+T494V) capsid variant. In some embodiments, the capsid comprises the amino acid sequence of SEQ ID NO: 6, which is provided below. SEQ ID NO: 6 corresponds to the

AAV8(Y444F + Y733F + T494V) capsid variant.
(SEQ ID NO: 6)
MAADGYLPDWLEDNLSEGIREWWALKPGAPKPKAN
QQKQDDGRGLVLPGYKYLGPFNGLDKGEPVNAADA
AALEHDKAYDQQLQAGDNPYLRYNHADAEFQERLQ
EDTSFGGNLGRAVFQAKKRVLEPLGLVEEGAKTAP
GKKRPVEPSPQRSPDSSTGIGKKGQQPARKRLNFG
QTGDSESVPDPQPLGEPPAAPSGVGPNTMAAGGGA
PMADNNEGADGVGSSSGNWHCDSTWLGDRVITTST
RTWALPTYNNHLYKQISNGTSGGATNDNTYFGYST
PWGYFDFNRFHCHFSPRDWQRLINNNWGFRPKRLS
FKLFNIQVKEVTQNEGTKTIANNLTSTIQVFTDSE
YQLPYVLGSAHQGCLPPFPADVFMIPQYGYLTLNN
GSQAVGRSSFYCLEYFPSQMLRTGNNFQFTYTFED
VPFHSSYAHSQSLDRLMNPLIDQFFLYYLSRTQTT
GGTANTQTLGFSQGGPNTMANQAKNWLPGPCYRQQ
RVSTVTGQNNNSNFAWTAGTKYHLNGRNSLANPGI
AMATHKDDEERFFPSNGILIFGKQNAARDNADYSD
VMLTSEEEIKTTNPVATEEYGIVADNLQQQNTAPQ
IGTVNSQGALPGMVWQNRDVYLQGPIWAKIPHTDG
NFHPSPLMGGFGLKHPPPQILIKNTPVPADPPTTF
NQSKLNSFITQYSTGQVSVEIEWELQKENSKRWNP
EIQYTSNYYKSTSVDFAVNTEGVYSEPRPIGTRFL
TRNL

In certain embodiments, the methods of treatment of the disclosure comprise administrations of the therapeutic vectors, particles and compositions to the brain of a subject, e.g., a mammalian subject such as a human subject. In some embodiments, the methods comprise intracranial, intraparenchymal, (intra)cisternal, intrathalamic, intrathoracic, intravenous, intramuscular, and intromyocardial administrations to a subject. Thalamic administrations of rAAV8 vectors are described in Gilkes, Bloom & Heldermon, Molecular Genetics and Metabolism Reports, 6 (2016) 48-54. In some embodiments, the methods comprise intracranial, cisternal, or intraparenchymal administrations. In some embodiments, the methods comprise one or more intraparenchymal injections into the bilateral frontal cortex, temporal cortex, hippocampus, caudate putamen, inferior colliculus, superior colliculus, thalamus and/or cerebellum of the subject. In particular embodiments, the methods comprise one or more intraparenchymal injections into the bilateral frontal cortex, temporal cortex, and/or cerebellum of the subject, e.g. an intraparenchymal 6-site injection (IC6). In some embodiments, the methods comprise cisternal magna injections.

In some embodiments of the methods of treatment provided herein, the methods do not comprise the co-administration of an agent with the rAAV particle (or vector). In some embodiments, the methods comprise the co-administration of an agent in combination with the rAAV particle (or vector). In some embodiments, the agent is a pharmaceutically-active agent, such as an immunomodulatory agent. In some embodiments, an adjuvant is co-administered with the rAAV particles, e.g., after administration of the rAAV particles.

As used herein, the terms “Sanfilippo syndrome type B,” “Sanfilippo syndrome B” and “Mucopolysaccharidoses IIIB” are used synonymously. Sanfilippo syndrome type B is typically used in the art to describe a lysosomal storage condition in humans. Mucopolysaccharidoses IIIB (MPS IIIB) is typically used in the art to describe the lysosomal storage condition in non-human mammals.

The disclosed vectors are adapted for delivery of a heterologous nucleic acid (or transgene) comprising NAGLU to mammalian cells, such as human cells. In certain embodiments, the heterologous nucleic acid comprises a coNAGLU cDNA sequence that has been codon-optimized for expression in human cells. In various embodiments, the heterologous nucleic acid encodes a protein that comprises the amino acid sequence of a wild-type NAGLU. In some embodiments, the heterologous nucleic acid encodes a protein that comprises an amino acid sequence that differs by 1, 2, 3, 4, or 5 amino acids, or between 5 and 10 amino acids, from the amino acid sequence of a wild-type NAGLU.

In some embodiments, the heterologous nucleic acid sequence has been further codon-modified, e.g., mutated to have one or more putative stop codons in non-coding sequences removed. In some embodiments, the nucleic acid sequence is codon-optimized for human expression and/or codon-modified.

In various embodiments, the disclosed rAAV vector comprises a heterologous nucleic acid encoding NAGLU, wherein the heterologous nucleic acid comprises a sequence having at least 85% identity, at least 90% identity, at least 92.5% identity, at least 95% identity, at least 98% identity, or at least 99% identity to the nucleic acid sequence of SEQ ID NO: 1. In some embodiments, the heterologous nucleic acid sequence has at least 90% identity the sequence of SEQ ID NO: 1. In some embodiments, the disclosed heterologous nucleic acid sequence comprises one or more truncations at the 5′ or 3′ end relative to SEQ ID NO: 1.

In particular embodiments, the heterologous nucleic acid comprises the sequence of SEQ ID NO: 1. This sequence is 2,264 nucleotides in length and is provided below.

(SEQ ID NO: 1)
ATGGAGGCTGTGGCTGTGGCTGCCGCTGTGGGAGT
GCTGCTGCTGGCTGGGGCCGGAGGGGCCGCTGGCG
ATGAGGCTCGGGAGGCTGCAGCTGTGCGCGCACTG
GTCGCACGACTGCTGGGACCTGGGCCAGCAGCCGA
CTTCTCTGTGAGTGTCGAGAGAGCCCTGGCTGCAA
AGCCCGGACTGGATACCTACAGTCTGGGAGGAGGA
GGAGCAGCTCGAGTGAGGGTCAGAGGGTCAACAGG
AGTGGCAGCAGCTGCAGGACTGCACCGATATCTGC
GAGACTTTTGCGGCTGTCATGTGGCCTGGTCAGGA
AGCCAGCTGAGGCTGCCCAGACCTCTGCCAGCAGT
GCCAGGCGAGCTGACAGAAGCCACTCCCAACCGGT
ACCGCTACTATCAGAACGTGTGCACCCAGTCCTAT
TCTTTCGTCTGGTGGGACTGGGCTCGATGGGAGCG
CGAAATCGATTGGATGGCACTGAACGGAATTAATC
TGGCACTGGCATGGTCCGGACAGGAGGCAATCTGG
CAGAGAGTGTACCTGGCACTGGGACTGACTCAGGC
CGAGATTAACGAGTTCTTCACCGGGCCAGCTTTTC
TGGCATGGGGACGGATGGGGAATCTGCACACATGG
GACGGACCACTGCCACCTTCTTGGCACATCAAACA
GCTGTATCTGCAGCATAGGGTGCTGGATCAGATGA
GAAGTTTTGGCATGACTCCAGTGCTGCCCGCTTTC
GCAGGACACGTCCCTGAGGCCGTGACACGCGTCTT
CCCACAGGTGAACGTCACTAAGATGGGCAGCTGGG
GACACTTCAACTGCAGTTACTCATGTAGCTTCCTG
CTGGCCCCTGAAGATCCAATTTTTCCCATCATTGG
AAGCCTGTTCCTGCGGGAGCTGATCAAAGAATTTG
GAACCGACCACATCTACGGGGCCGATACATTCAAC
GAGATGCAGCCACCCAGCTCCGAACCTAGCTACCT
GGCCGCTGCAACCACAGCCGTGTACGAGGCCATGA
CCGCTGTGGACACAGAAGCCGTCTGGCTGCTGCAG
GGGTGGCTGTTTCAGCATCAGCCACAGTTCTGGGG
ACCTGCACAGATCCGAGCTGTGCTGGGAGCAGTCC
CACGAGGAAGGCTGCTGGTGCTGGATCTGTTCGCT
GAGTCCCAGCCCGTCTACACTAGGACCGCCTCTTT
CCAGGGCCAGCCTTTTATTTGGTGTATGCTGCACA
ACTTTGGAGGGAATCATGGGCTGTTCGGCGCACTG
GAGGCAGTGAACGGAGGACCAGAAGCAGCTAGACT
GTTTCCTAATAGCACTATGGTGGGCACCGGAATGG
CTCCCGAGGGCATCTCACAGAATGAAGTGGTCTAC
AGCCTGATGGCAGAGCTGGGATGGCGAAAGGACCC
CGTGCCTGATCTGGCAGCCTGGGTCACTAGTTTCG
CTGCAAGGAGATACGGGGTGTCACACCCTGACGCT
GGAGCAGCTTGGCGACTGCTGCTGAGGAGCGTGTA
CAACTGCAGTGGGGAGGCCTGTAGAGGCCATAATC
GGTCCCCACTGGTGCGGCGCCCCTCACTGCAGATG
AACACCAGCATCTGGTACAATCGATCCGACGTGTT
CGAAGCTTGGCGGCTGCTGCTGACAAGTGCCCCTT
CACTGGCTACTTCTCCAGCATTCCGCTATGACCTG
CTGGATCTGACAAGGCAGGCCGTGCAGGAGCTGGT
CAGCCTGTACTATGAGGAAGCTCGCAGCGCATACC
TGTCCAAGGAACTGGCATCCCTGCTGAGGGCAGGA
GGCGTGCTGGCTTATGAGCTGCTGCCAGCTCTGGA
CGAAGTCCTGGCATCCGATTCTAGATTTCTGCTGG
GCAGCTGGCTGGAGCAGGCACGAGCAGCAGCCGTG
AGCGAGGCCGAAGCTGACTTCTACGAGCAGAACTC
TAGGTATCAGCTGACTCTGTGGGGACCCGAAGGGA
ACATCCTGGATTACGCAAACAAGCAGCTGGCAGGA
CTGGTGGCTAATTACTATACCCCTAGATGGCGGCT
GTTTCTGGAGGCCCTGGTGGACTCTGTCGCTCAGG
GCATTCCATTCCAGCAGCACCAGTTTGATAAGAAC
GTGTTCCAGCTGGAACAGGCCTTCGTCCTGTCTAA
ACAGCGGTATCCTAGTCAGCCACGCGGCGACACAG
TGGACCTGGCAAAGAAGATTTTCCTGAAATACTAT
CCCCGCTGGGTGGCTGGCTCATGGACCGGTGGCGC
GCCTTCGAACTGCAGCTCGAGGTA

Accordingly, in some embodiments, the disclosed rAAV vector comprises a heterologous nucleic acid encoding NAGLU, wherein the heterologous nucleic acid comprises a sequence having at least 85% identity, at least 90% identity, at least 92.5% identity, at least 95% identity, at least 98% identity, or at least 99% identity to the nucleic acid sequence of SEQ ID NO: 1, wherein the rAAV vector is of serotype 8, or a variant thereof. In some embodiments, the disclosed rAAV vector comprises a heterologous nucleic acid encoding NAGLU, wherein the heterologous nucleic acid comprises the nucleic acid sequence of SEQ ID NO: 1, and the vector is of serotype variant AAV8(Y447F+Y733F+T494V).

In various embodiments, the disclosed rAAV vector comprises a heterologous nucleic acid that comprises a sequence having at least 85% identity, at least 90% identity, at least 92.5% identity, at least 95% identity, at least 98% identity, or at least 99% identity to the nucleic acid sequence of SEQ ID NO: 7. In some embodiments, the heterologous nucleic acid sequence has at least 90% identity the sequence of SEQ ID NO: 7. In some embodiments, the disclosed heterologous nucleic acid sequence comprises one or more truncations at the 5′ or 3′ end relative to SEQ ID NO: 7. SEQ ID NO: 7 represents a NAGLU sequence that has been codon-optimized for expression in human cells.

In particular embodiments, the heterologous nucleic acid comprises the sequence of SEQ ID NO: 7. This sequence is 2,232 nucleotides in length and is provided below.

(SEQ ID NO: 7)
ATGGAGGCTGTGGCTGTGGCTGCCGCTGTGGGAGT
GCTGCTGCTGGCTGGGGCCGGAGGGGCCGCTGGCG
ATGAGGCTCGGGAGGCTGCAGCTGTGCGCGCACTG
GTCGCACGACTGCTGGGACCTGGGCCAGCAGCCGA
CTTCTCTGTGAGTGTCGAGAGAGCCCTGGCTGCAA
AGCCCGGACTGGATACCTACAGTCTGGGAGGAGGA
GGAGCAGCTCGAGTGAGGGTCAGAGGGTCAACAGG
AGTGGCAGCAGCTGCAGGACTGCACCGATATCTGC
GAGACTTTTGCGGCTGTCATGTGGCCTGGTCAGGA
AGCCAGCTGAGGCTGCCCAGACCTCTGCCAGCAGT
GCCAGGCGAGCTGACAGAAGCCACTCCCAACCGGT
ACCGCTACTATCAGAACGTGTGCACCCAGTCCTAT
TCTTTCGTCTGGTGGGACTGGGCTCGATGGGAGCG
CGAAATCGATTGGATGGCACTGAACGGAATTAATC
TGGCACTGGCATGGTCCGGACAGGAGGCAATCTGG
CAGAGAGTGTACCTGGCACTGGGACTGACTCAGGC
CGAGATTAACGAGTTCTTCACCGGGCCAGCTTTTC
TGGCATGGGGACGGATGGGGAATCTGCACACATGG
GACGGACCACTGCCACCTTCTTGGCACATCAAACA
GCTGTATCTGCAGCATAGGGTGCTGGATCAGATGA
GAAGTTTTGGCATGACTCCAGTGCTGCCCGCTTTC
GCAGGACACGTCCCTGAGGCCGTGACACGCGTCTT
CCCACAGGTGAACGTCACTAAGATGGGCAGCTGGG
GACACTTCAACTGCAGTTACTCATGTAGCTTCCTG
CTGGCCCCTGAAGATCCAATTTTTCCCATCATTGG
AAGCCTGTTCCTGCGGGAGCTGATCAAAGAATTTG
GAACCGACCACATCTACGGGGCCGATACATTCAAC
GAGATGCAGCCACCCAGCTCCGAACCTAGCTACCT
GGCCGCTGCAACCACAGCCGTGTACGAGGCCATGA
CCGCTGTGGACACAGAAGCCGTCTGGCTGCTGCAG
GGGTGGCTGTTTCAGCATCAGCCACAGTTCTGGGG
ACCTGCACAGATCCGAGCTGTGCTGGGAGCAGTCC
CACGAGGAAGGCTGCTGGTGCTGGATCTGTTCGCT
GAGTCCCAGCCCGTCTACACTAGGACCGCCTCTTT
CCAGGGCCAGCCTTTTATTTGGTGTATGCTGCACA
ACTTTGGAGGGAATCATGGGCTGTTCGGCGCACTG
GAGGCAGTGAACGGAGGACCAGAAGCAGCTAGACT
GTTTCCTAATAGCACTATGGTGGGCACCGGAATGG
CTCCCGAGGGCATCTCACAGAATGAAGTGGTCTAC
AGCCTGATGGCAGAGCTGGGATGGCGAAAGGACCC
CGTGCCTGATCTGGCAGCCTGGGTCACTAGTTTCG
CTGCAAGGAGATACGGGGTGTCACACCCTGACGCT
GGAGCAGCTTGGCGACTGCTGCTGAGGAGCGTGTA
CAACTGCAGTGGGGAGGCCTGTAGAGGCCATAATC
GGTCCCCACTGGTGCGGCGCCCCTCACTGCAGATG
AACACCAGCATCTGGTACAATCGATCCGACGTGTT
CGAAGCTTGGCGGCTGCTGCTGACAAGTGCCCCTT
CACTGGCTACTTCTCCAGCATTCCGCTATGACCTG
CTGGATCTGACAAGGCAGGCCGTGCAGGAGCTGGT
CAGCCTGTACTATGAGGAAGCTCGCAGCGCATACC
TGTCCAAGGAACTGGCATCCCTGCTGAGGGCAGGA
GGCGTGCTGGCTTATGAGCTGCTGCCAGCTCTGGA
CGAAGTCCTGGCATCCGATTCTAGATTTCTGCTGG
GCAGCTGGCTGGAGCAGGCACGAGCAGCAGCCGTG
AGCGAGGCCGAAGCTGACTTCTACGAGCAGAACTC
TAGGTATCAGCTGACTCTGTGGGGACCCGAAGGGA
ACATCCTGGATTACGCAAACAAGCAGCTGGCAGGA
CTGGTGGCTAATTACTATACCCCTAGATGGCGGCT
GTTTCTGGAGGCCCTGGTGGACTCTGTCGCTCAGG
GCATTCCATTCCAGCAGCACCAGTTTGATAAGAAC
GTGTTCCAGCTGGAACAGGCCTTCGTCCTGTCTAA
ACAGCGGTATCCTAGTCAGCCACGCGGCGACACAG
TGGACCTGGCAAAGAAGATTTTCCTGAAATACTAT
CCCCGCTGGGTGGCTGGCTCATGGTAA

In some embodiments, any of the disclosed vectors comprise a linker sequence within the coNAGLU sequence. In particular embodiments, the disclosed vectors do not contain a linker sequence within the coNAGLU sequence.

In some embodiments, any of the disclosed rAAV vectors comprises a promoter that is operably linked to, and operably controls, the coNAGLU heterologous nucleic acid. The promoters may comprise any one of a chicken beta actin (CBA) promoter, cytomegalovirus promoter, chimeric CBA-cytomegalovirus promoter, truncated chimeric CBA-cytomegalovirus promoter, transthyretin promoter, desmin promoter, PGK promoter, GAD67 promoter, Dl×5/6 promoter, Drd1a promoter, SYN promoter, GFAP promoter, MAP2 promoter, or another promoter. The promoter may comprise a hybrid promoter, such as a hybrid CMV enhancer/chicken β-actin (CAG) promoter. The promoter may comprise a neuronal tissue-specific promoter. The promoter may mediate or enhance expression in brain tissues, such as neurons. In particular embodiments, the promoter is a CBA promoter.

The heterologous nucleic acid of the disclosed rAAV vectors may be flanked by two AAV inverted terminal repeat (ITR) sequences. The ITR sequences of the disclosed rAAV vectors may be derived from any AAV serotype (e.g., 1, 2, 3, 4, 5, 6, 7, 8, 9, or 10) or may be from more than one serotype. In some embodiments, the ITR sequences are from AAV2. In some embodiments, the ITR sequences are wild-type AAV2 ITR sequences. In particular embodiments, the rAAV vectors comprise coNAGLU flanked by wild-type AAV2 ITRs.

Accordingly, in particular embodiments, the disclosure provides an rAAV vector comprising a CBA promoter operably linked to a coNAGLU heterologous nucleic acid, wherein the AAV vector comprises AAV2 ITRs. In particular embodiments, the disclosure provides an rAAV vector comprising a CBA promoter operably linked to a coNAGLU heterologous nucleic acid, wherein the AAV vector comprises AAV2 ITRs and is of a serotype variant AAV8(Y447F+Y733F+T494V).

The rAAV vectors of the disclosure may comprise a coNAGLU sequence flanked by ITRs, e.g., AAV2 ITRs. In some embodiments, the disclosed rAAV vectors comprise a nucleotide sequence that has at least 80% identity, at least 85% identity, at least 90% identity, at least 92.5% identity, at least 95% identity, at least 98% identity, or at least 99% identity to the sequence of SEQ ID NO: 2. In some embodiments, the rAAV vector nucleotide sequence has at least 85% identity the sequence of SEQ ID NO: 2. In particular embodiments, the rAAV vector nucleotide sequence has at least 95% identity the sequence of SEQ ID NO: 2. In some embodiments, the rAAV vector comprises the ITRs, promoter, coNAGLU heterologous nucleic acid, and terminator comprised in SEQ ID NO: 2.

In particular embodiments, the disclosed rAAV vector comprises a nucleotide sequence that comprises the sequence of SEQ ID NO: 2. The vector of SEQ ID NO: 2 is referred to herein as AAVtcm8-coNAGLU, AAVtcm8-NAGLU, or tcm8-coNAGLU. This sequence is 4,533 nucleotides in length and is provided below.

(SEQ ID NO: 2)
TTGGCCACTCCCTCTCTGCGCGCTCGCTCGCTCAC
TGAGGCCGGGCGACCAAAGGTCGCCCGACGCCCGG
GCTTTGCCCGGGCGGCCTCAGTGAGCGAGCGAGCG
CGCAGAGAGGGAGTGGCCAACTCCATCACTAGGGG
TTCCTCAGATCTGAATTCGGTACCTAGTTATTAAT
AGTAATCAATTACGGGGTCATTAGTTCATAGCCCA
TATATGGAGTTCCGCGTTACATAACTTACGGTAAA
TGGCCCGCCTGGCTGACCGCCCAACGACCCCCGCC
CATTGACGTCAATAATGACGTATGTTCCCATAGTA
ACGCCAATAGGGACTTTCCATTGACGTCAATGGGT
GGAGTATTTACGGTAAACTGCCCACTTGGCAGTAC
ATCAAGTGTATCATATGCCAAGTACGCCCCCTATT
GACGTCAATGACGGTAAATGGCCCGCCTGGCATTA
TGCCCAGTACATGACCTTATGGGACTTTCCTACTT
GGCAGTACATCTACGTATTAGTCATCGCTATTACC
ATGGTCGAGGTGAGCCCCACGTTCTGCTTCACTCT
CCCCATCTCCCCCCCCTCCCCACCCCCAATTTTGT
ATTTATTTATTTTTTAATTATTTTGTGCAGCGATG
GGGGCGGGGGGGGGGGGGGGGCGCGCGCCAGGCGG
GGCGGGGCGGGGCGAGGGGCGGGGCGGGGCGAGGC
GGAGAGGTGCGGCGGCAGCCAATCAGAGCGGCGCG
CTCCGAAAGTTTCCTTTTATGGCGAGGCGGCGGCG
GCGGCGGCCCTATAAAAAGCGAAGCGCGCGGCGGG
CGGGAGTCGCTGCGCGCTGCCTTCGCCCCGTGCCC
CGCTCCGCCGCCGCCTCGCGCCGCCCGCCCCGGCT
CTGACTGACCGCGTTACTCCCACAGGTGAGCGGGC
GGGACGGCCCTTCTCCTCCGGGCTGTAATTAGCGC
TTGGTTTAATGACGGCTTGTTTCTTTTCTGTGGCT
GCGTGAAAGCCTTGAGGGGCTCCGGGAGGGCCCTT
TGTGCGGGGGGAGCGGCTCGGGGGGTGCGTGCGTG
TGTGTGTGCGTGGGGAGCGCCGCGTGCGGCTCCGC
GCTGCCCGGCGGCTGTGAGCGCTGCGGGCGCGGCG
CGGGGCTTTGTGCGCTCCGCAGTGTGCGCGAGGGG
AGCGCGGCCGGGGGCGGTGCCCCGCGGTGCGGGGG
GGGCTGCGAGGGGAACAAAGGCTGCGTGCGGGGTG
TGTGCGTGGGGGGGTGAGCAGGGGGTGTGGGCGCG
TCGGTCGGGCTGCAACCCCCCCTGCACCCCCCTCC
CCGAGTTGCTGAGCACGGCCCGGCTTCGGGTGCGG
GGCTCCGTACGGGGCGTGGCGCGGGGCTCGCCGTG
CCGGGCGGGGGGTGGCGGCAGGTGGGGGTGCCGGG
CGGGGCGGGGCCGCCTCGGGCCGGGGAGGGCTCGG
GGGAGGGGCGCGGCGGCCCCCGGAGCGCCGGCGGC
TGTCGAGGCGCGGCGAGCCGCAGCCATTGCCTTTT
ATGGTAATCGTGCGAGAGGGCGCAGGGACTTCCTT
TGTCCCAAATCTGTGCGGAGCCGAAATCTGGGAGG
CGCCGCCGCACCCCCTCTAGCGGGCGCGGGGCGAA
GCGGTGCGGCGCCGGCAGGAAGGAAATGGGCGGGG
AGGGCCTTCGTGCGTCGCCGCGCCGCCGTCCCCTT
CTCCCTCTCCAGCCTCGGGGCTGTCCGCGGGGGGA
CGGCTGCCTTCGGGGGGGACGGGGCAGGGCGGGGT
TCGGCTTCTGGCGTGTGACCGGCGGCTCTAGAGCC
TCTGCTAACCATGTTCATGCCTTCTTCTTTTTCCT
ACAGCTCCTGGGCAACGTGCTGGTTATTGTGCTGT
CTCATCATTTTGGCAAAGAATTCCTCGAAGATCTA
GGCCTGCAGGCGGCCGCCACCATGGAGGCTGTGGC
TGTGGCTGCCGCTGTGGGAGTGCTGCTGCTGGCTG
GGGCCGGAGGGGCCGCTGGCGATGAGGCTCGGGAG
GCTGCAGCTGTGCGCGCACTGGTCGCACGACTGCT
GGGACCTGGGCCAGCAGCCGACTTCTCTGTGAGTG
TCGAGAGAGCCCTGGCTGCAAAGCCCGGACTGGAT
ACCTACAGTCTGGGAGGAGGAGGAGCAGCTCGAGT
GAGGGTCAGAGGGTCAACAGGAGTGGCAGCAGCTG
CAGGACTGCACCGATATCTGCGAGACTTTTGCGGC
TGTCATGTGGCCTGGTCAGGAAGCCAGCTGAGGCT
GCCCAGACCTCTGCCAGCAGTGCCAGGCGAGCTGA
CAGAAGCCACTCCCAACCGGTACCGCTACTATCAG
AACGTGTGCACCCAGTCCTATTCTTTCGTCTGGTG
GGACTGGGCTCGATGGGAGCGCGAAATCGATTGGA
TGGCACTGAACGGAATTAATCTGGCACTGGCATGG
TCCGGACAGGAGGCAATCTGGCAGAGAGTGTACCT
GGCACTGGGACTGACTCAGGCCGAGATTAACGAGT
TCTTCACCGGGCCAGCTTTTCTGGCATGGGGACGG
ATGGGGAATCTGCACACATGGGACGGACCACTGCC
ACCTTCTTGGCACATCAAACAGCTGTATCTGCAGC
ATAGGGTGCTGGATCAGATGAGAAGTTTTGGCATG
ACTCCAGTGCTGCCCGCTTTCGCAGGACACGTCCC
TGAGGCCGTGACACGCGTCTTCCCACAGGTGAACG
TCACTAAGATGGGCAGCTGGGGACACTTCAACTGC
AGTTACTCATGTAGCTTCCTGCTGGCCCCTGAAGA
TCCAATTTTTCCCATCATTGGAAGCCTGTTCCTGC
GGGAGCTGATCAAAGAATTTGGAACCGACCACATC
TACGGGGCCGATACATTCAACGAGATGCAGCCACC
CAGCTCCGAACCTAGCTACCTGGCCGCTGCAACCA
CAGCCGTGTACGAGGCCATGACCGCTGTGGACACA
GAAGCCGTCTGGCTGCTGCAGGGGTGGCTGTTTCA
GCATCAGCCACAGTTCTGGGGACCTGCACAGATCC
GAGCTGTGCTGGGAGCAGTCCCACGAGGAAGGCTG
CTGGTGCTGGATCTGTTCGCTGAGTCCCAGCCCGT
CTACACTAGGACCGCCTCTTTCCAGGGCCAGCCTT
TTATTTGGTGTATGCTGCACAACTTTGGAGGGAAT
CATGGGCTGTTCGGCGCACTGGAGGCAGTGAACGG
AGGACCAGAAGCAGCTAGACTGTTTCCTAATAGCA
CTATGGTGGGCACCGGAATGGCTCCCGAGGGCATC
TCACAGAATGAAGTGGTCTACAGCCTGATGGCAGA
GCTGGGATGGCGAAAGGACCCCGTGCCTGATCTGG
CAGCCTGGGTCACTAGTTTCGCTGCAAGGAGATAC
GGGGTGTCACACCCTGACGCTGGAGCAGCTTGGCG
ACTGCTGCTGAGGAGCGTGTACAACTGCAGTGGGG
AGGCCTGTAGAGGCCATAATCGGTCCCCACTGGTG
CGGCGCCCCTCACTGCAGATGAACACCAGCATCTG
GTACAATCGATCCGACGTGTTCGAAGCTTGGCGGC
TGCTGCTGACAAGTGCCCCTTCACTGGCTACTTCT
CCAGCATTCCGCTATGACCTGCTGGATCTGACAAG
GCAGGCCGTGCAGGAGCTGGTCAGCCTGTACTATG
AGGAAGCTCGCAGCGCATACCTGTCCAAGGAACTG
GCATCCCTGCTGAGGGCAGGAGGCGTGCTGGCTTA
TGAGCTGCTGCCAGCTCTGGACGAAGTCCTGGCAT
CCGATTCTAGATTTCTGCTGGGCAGCTGGCTGGAG
CAGGCACGAGCAGCAGCCGTGAGCGAGGCCGAAGC
TGACTTCTACGAGCAGAACTCTAGGTATCAGCTGA
CTCTGTGGGGACCCGAAGGGAACATCCTGGATTAC
GCAAACAAGCAGCTGGCAGGACTGGTGGCTAATTA
CTATACCCCTAGATGGCGGCTGTTTCTGGAGGCCC
TGGTGGACTCTGTCGCTCAGGGCATTCCATTCCAG
CAGCACCAGTTTGATAAGAACGTGTTCCAGCTGGA
ACAGGCCTTCGTCCTGTCTAAACAGCGGTATCCTA
GTCAGCCACGCGGCGACACAGTGGACCTGGCAAAG
AAGATTTTCCTGAAATACTATCCCCGCTGGGTGGC
TGGCTCATGGTAAGTCGACTAGAGCTCGCTGATCA
GCCTCGACTGTGCCTTCTAGTTGCCAGCCATCTGT
TGTTTGCCCCTCCCCCGTGCCTTCCTTGACCCTGG
AAGGTGCCACTCCCACTGTCCTTTCCTAATAAAAT
GAGGAAATTGCATCGCATTGTCTGAGTAGGTGTCA
TTCTATTCTGGGGGGTGGGGTGGGGCAGGACAGCA
AGGGGGAGGATTGGGAAGACAATAGCAGGCATGCT
GGGGAGAGATCTGAGGAACCCCTAGTGATGGAGTT
GGCCACTCCCTCTCTGCGCGCTCGCTCGCTCACTG
AGGCCGCCCGGGCAAAGCCCGGGCGTCGGGCGACC
TTTGGTCGCCCGGCCTCAGTGAGCGAGCGAGCGCG
CAGAGAGGGAGTGGCCAA

By a nucleic acid molecule (e.g., a rAAV vector sequence or a NAGLU sequence) comprising a nucleotide sequence having at least, for example, 95% “identity” to a query nucleic acid sequence, it is intended that the nucleotide sequence of the subject nucleic acid molecule is identical to the query sequence except that the subject nucleic acid molecule sequence may include up to five nucleotide alterations per each 100 nucleotides of the query sequence. In other words, to obtain a promoter having a nucleotide sequence at least 95% identical to a reference (query) sequence, up to 5% of the nucleotides in the subject sequence may be inserted, deleted, or substituted with another nucleotide. These alterations of the reference sequence may occur at the 5′ or 3′ ends of the reference sequence or anywhere between those positions, interspersed either individually among nucleotides in the reference sequence or in one or more contiguous groups within the reference sequence.

As a practical matter, whether any particular nucleic acid molecule is at least 80%, 85%, 90%, 95%, 96%, 97%, 98%, or 99% identical to, for instance, the nucleotide sequence of a NAGLU sequence, can be determined conventionally using known computer programs. A preferred method for determining the best overall match between a query sequence (e.g., a sequence of the present disclosure) and a subject sequence, also referred to as a global sequence alignment, can be determined using the FASTDB or blastn computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter. Whether a nucleotide is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present disclosure. For subject sequences truncated at the 5′ and/or 3′ ends, relative to the query sequence, the percent identity is corrected by calculating the number of nucleotides of the query sequence that are positioned 5′ to or 3′ to the query sequence, which are not matched/aligned with a corresponding subject nucleotide, as a percent of the total bases of the query sequence.

In some embodiments, the rAAV vectors described herein may comprise a heterologous nucleic acid comprising a sequence having 1, 2, 3, 4, 5, 6, 7, 8, 9, 10, 11, 12, 13, 14, 15, 16, 17, or 18 nucleotides that differ relative to the NAGLU sequence as set forth in SEQ ID NO: 1 or 7. The heterologous nucleic acids of the disclosure may comprise 1, 2, 3, 4, 5, 6, 7, 8, 9, 10, 11, 12 or more than 12 silent mutations that do not result in mutations in the encoded NAGLU protein sequence relative to SEQ ID NO: 1 or 7. In some embodiments, the protein coding sequence of SEQ ID NO: 1 or SEQ ID NO: 7 is comprised within an rAAV vector. In some embodiments, a sequence comprising 85%, 90%, 95%, 98%, 99% or greater than 99% identity to the protein coding sequence of SEQ ID NO: 1 or 7 is comprised within an rAAV vector. In some embodiments, the disclosed rAAV vectors comprise a sequence comprising the nucleotides of positions 1 to 2232 or 1 to 2262 of SEQ ID NO: 1.

In some embodiments, the rAAV vectors described herein may comprise a nucleic acid sequence having 1, 2, 3, 4, 5, 6, 7, 8, 9, 10, 11, 12, 13, 14, 15, 16, 17, 18, 19, 20, 21-25, 25-30, 30-35, 35-40, or more than 40 nucleotides that differ relative to the vector sequence as set forth in SEQ ID NO: 2. These differences may comprise nucleotides that have been inserted, deleted, or substituted relative to the sequence of SEQ ID NOs: 1, 2 or 7. In some embodiments, the rAAV vectors comprise sequences that contain truncations of 1, 2, 3, 4, 5, 6, 7, 8, 9, 10 or more than 10 nucleotides at the 5′ or 3′ end relative to SEQ ID NOs: 1, 2 or 7. In some embodiments, the disclosed rAAV vectors comprise sequences that contain stretches of about 50, about 75, about 100, about 125, about 150, about 175, or about 180 nucleotides in common with the sequence of SEQ ID NOs: 1, 2 or 7. In some embodiments, the disclosed polynucleotides contain stretches of about 200, about 300, about 400, about 500, about 600, about 700, about 800, about 900, about 1000, or more than about 1000 nucleotides in common with the sequence of SEQ ID NO: 2.

Further aspects of this disclosure relate to methods of transducing a host cell comprising administering an effective amount of the rAAV particles or the pharmaceutical compositions disclosed herein to the cell. Any host cell is contemplated for use in a method described herein. In some embodiments, the host cell is a cell in situ in a host, such as a subject as described herein. In some embodiments, the host cell is ex vivo, e.g., in a culture of host cells. In particular embodiments, the host cell is a brain cell, such as a neuron or glia. In some embodiments, the host cell is a cell of the cerebral cortex, thalamus, hippocampus or cerebellum.

### Pharmaceutical Compositions

As described herein, further provided herein are pharmaceutical compositions that comprise a modified rAAV vector as disclosed herein, and further comprise one or more pharmaceutical excipients. These compositions may be formulated for administration to host cells ex vivo or in situ in an animal, and particularly a human. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Such compositions may be formulated for use in a variety of therapies, such as for example, in the amelioration, prevention, and/or treatment of conditions such as peptide deficiency, polypeptide deficiency, peptide overexpression, polypeptide overexpression, including for example, conditions, diseases or disorders as described herein. In particular embodiments, the described compositions may be formulated for use in the amelioration, prevention, and/or treatment of Sanfilippo syndrome, type B.

The term “excipient” refers to a diluent, adjuvant, carrier, or vehicle with which the rAAV particle or preparation, or nucleic acid vector is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers.

In certain embodiments, the present disclosure provides a method of reducing AAV immunity in a subject, wherein the method further comprises administering to the subject a composition comprising the disclosed rAAV particles and a pharmaceutically acceptable excipient, optionally wherein the subject has been previously administered a composition comprising rAAV particles. In particular embodiments, the subject is a human. In certain embodiments, the subject is suspected of having, is at risk for developing, or has been diagnosed with Sanfilippo syndrome.

In some embodiments, the number of rAAV particles administered to a subject may be on the order ranging from 10⁶ to 10¹⁴ particles/mL or 10³ to 10¹³ particles/mL, or any values therebetween for either range, such as for example, about 10⁶, 10⁷, 10⁸, 10⁹, 10¹⁰, 10¹¹, 10¹², 10¹³, or 10¹⁴ particles/mL. In one embodiment, rAAV particles of higher than 10¹³ particles/mL are be administered. In some embodiments, the number of rAAV particles administered to a subject may be on the order ranging from 10⁶ to 10¹⁴ vector genomes(vgs)/mL or 10³ to 10¹⁵ vgs/mL, or any values there between for either range, such as for example, about 10⁶, 10⁷, 10⁸, 10⁹, 10¹⁰, 10¹¹, 10¹², 10¹³, or 10¹⁴ vgs/mL. In certain embodiments, the methods comprise administration of compositions comprising rAAV particle compositions in doses of 3×10⁹ to 3×10¹⁰ vgs/mL. In certain embodiments, the methods comprise administration of compositions comprising rAAV particle compositions in a dose of 1.8×10¹⁰ vgs/mL.

In some embodiments, the amount of AAV particles administered to a subject may be on the order ranging from 10⁶ to 10¹⁴ particles/ml or 10³ to 10¹⁵ particles/ml, or any values therebetween for either range, such as for example, about 10⁶, 10⁷, 10⁸, 10⁹, 10¹⁰, 10¹¹, 10¹², 10¹³, or 10¹⁴ particles/ml. In some embodiments, AAV particles of a higher concentration than 10¹³ particles/ml are administered. In some embodiments, the concentration of AAV particles administered to a subject may be on the order ranging from 10⁶ to 10¹⁴ vector genomes (vgs)/ml or 10³ to 10¹⁵ vgs/ml, or any values therebetween for either range (e.g., 10⁶, 10⁷, 10⁸, 10⁹, 10¹⁰, 10¹¹, 10¹², 10¹³, or 10¹⁴ vgs/ml). In some embodiments, AAV particles of higher concentration than 10¹³ vgs/ml are administered. In some embodiments, 0.0001 ml to 10 ml are delivered (e.g., via one or more routes of administration as described herein) to a subject. In some embodiments, the number of AAV particles administered to a subject may be on the order ranging from 10⁶-10¹⁴ vgs/kg body mass of the subject, or any values therebetween (e.g., 10⁶, 10⁷, 10⁸, 10⁹, 10¹⁰, 10¹¹, 10¹², 10¹³, or 10¹⁴ vgs/kg). In some embodiments, the dose of AAV particles administered to a subject may be on the order ranging from 10¹²-10¹⁴ vgs/kg.

The rAAV particles can be administered as a single dose or divided into two or more administrations as may be required to achieve therapy of the particular disease or disorder being treated (e.g., Sanfilippo syndrome B). In some embodiments, 0.0001 mL to 10 mLs are delivered to a subject.

In some embodiments, where a second nucleic acid vector encoding the Rep protein within a second rAAV particle is administered to a subject, the ratio of the first rAAV particle to the second rAAV particle is 1:100, 1:50, 1:40, 1:30, 1:20, 1:10, 1:5, 1:2 or 1:1. In some embodiments, the Rep protein is delivered to a subject such that target cells within the subject receive at least two Rep proteins per cell.

In some embodiments, the disclosure provides formulations of compositions disclosed herein in pharmaceutically acceptable solutions for administration to a cell or an animal, either alone or in combination with one or more other modalities of therapy, and in particular, for therapy of human cells, tissues, and diseases affecting man.

If desired, rAAV particle or preparation, Rep proteins, and nucleic acid vectors may be administered in combination with other agents as well, such as, e.g., proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The rAAV particles or preparations, Rep proteins, and nucleic acid vectors may thus be delivered along with various other agents as required in the particular instance. Such compositions may be purified from host cells or other biological sources, or alternatively may be chemically synthesized as described herein.

Formulation of pharmaceutically acceptable excipients is well-known to those of skill in the art, as is the development of suitable dosing and treatment regimens for using the particular compositions described herein in a variety of treatment regimens, including e.g., intracranial, intraparenchymal, (intra)cisternal, intrathalamic, intrathoracic, intravenous, intramuscular, and intromyocardial administration and formulation.

Typically, these formulations may contain at least about 0.1% of the therapeutic agent (e.g., rAAV particle or preparation, Rep protein, and/or nucleic acid vector) or more, although the percentage of the active ingredient(s) may, of course, be varied and may conveniently be between about 1 or 2% and about 70% or 80% or more of the weight or volume of the total formulation. Naturally, the amount of therapeutic agent(s) in each therapeutically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV particles or preparations, Rep proteins, and/or nucleic acid vectors in suitably formulated pharmaceutical compositions disclosed herein either subcutaneously intracranially, intraparenchymally, (intra)cisternally, intrathalamically, intrathoracically, intravenously, intromyocardially, intracerebro-ventricularly, intramuscularly, intrathecally, orally, intraperitoneally, by oral or nasal inhalation, or by direct injection to one or more cells, tissues, or organs by direct injection.

The pharmaceutical forms of the compositions suitable for injectable use include sterile aqueous solutions or dispersions. In some embodiments, the form is sterile and fluid to the extent that easy syringability exists. In some embodiments, the form is stable under the conditions of manufacture and storage and is preserved against the contaminating action of microorganisms, such as bacteria and fungi. The carrier can be a solvent or dispersion medium containing, for example, water, saline, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants.

The pharmaceutical compositions of the present disclosure can be administered to the subject being treated by standard routes including, but not limited to, intracranial, intraparenchymal, intracisternal, intrathalamic, intrathoracic, intravenous, intramuscular, intracerebro-ventricular, intrathecal, intrathoracic, VTA, and intromyocardial injection.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subcutaneous and intraperitoneal administration. In this connection, a sterile aqueous medium that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions are prepared by incorporating the rAAV particles or preparations, Rep proteins, and/or nucleic acid vectors, in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Ex vivo delivery of cells transduced with rAAV particles or preparations, and/or Rep proteins is also contemplated herein. Ex vivo gene delivery may be used to transplant rAAV-transduced host cells back into the host. A suitable ex vivo protocol may include several steps. For example, a segment of target tissue or an aliquot of target fluid may be harvested from the host and rAAV particles or preparations, and/or Rep proteins may be used to transduce a nucleic acid vector into the host cells in the tissue or fluid. These genetically modified cells may then be transplanted back into the host. Several approaches may be used for the reintroduction of cells into the host, including intravenous injection, intraperitoneal injection, or in situ injection into target tissue. Autologous and allogeneic cell transplantation may be used according to the disclosure.

In some embodiments, a composition disclosed herein (e.g., comprising an rAAV particle or nucleic acid of the disclosure) is administered to a subject once. In some embodiments, the composition is administered to a subject multiple times (e.g., twice, three times, four times, five times, six times, or more). Repeated administration to a subject may be conducted at a regular interval (e.g., daily, every other day, twice per week, weekly, twice per month, monthly, every six months, once per year, or less or more frequently) as necessary to treat (e.g., improve or alleviate) one or more symptoms of a disease, disorder, or condition in the subject (e.g., Sanfilippo syndrome).

In some embodiments, one or more cells isolated from a subject are contacted with an rAAV particle of the disclosure. In some embodiments, these cells are subsequently administered to a subject, e.g. the same subject from which the cell was isolated.

The amount of rAAV particle or preparation, Rep protein, or nucleic acid vector compositions and time of administration of such compositions will be within the purview of the skilled artisan having benefit of the present teachings. It is likely, however, that the administration of therapeutically-effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the rAAV particle or preparation, Rep protein, or nucleic acid vector compositions, either over a relatively short, or a relatively prolonged period of time, as may be determined by the medical practitioner overseeing the administration of such compositions.

To “treat” a disease as the term is used herein, means to reduce the frequency or severity of at least one sign or symptom of a disease or disorder experienced by a subject. The compositions described above are typically administered to a subject in an effective amount, that is, an amount capable of producing a desirable result. The desirable result will depend upon the active agent being administered. For example, an effective amount of a rAAV particle may be an amount of the particle that is capable of transferring a heterologous nucleic acid to a host organ, tissue, or cell. As used herein, to “treat” embraces preventing or inhibiting progression of a disease in a mammal, such as a human (e.g., Sanfilippo syndrome type B (or MPS IIIB)).

Toxicity and efficacy of the compositions utilized in methods of the disclosure can be determined by standard pharmaceutical procedures, using either cells in culture or experimental animals to determine the LD₅₀ (the dose lethal to 50% of the population). The dose ratio between toxicity and efficacy the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of compositions as described herein lies generally within a range that includes an ED₅₀ with little or no toxicity. The dosage may vary within this range depending upon the dosage form employed and the route of administration utilized.

Recombinant AAV (rAAV) Particles and Nucleic Acid Vectors

Aspects of the disclosure relate to recombinant adeno-associated virus (rAAV) particles or preparations of such particles for delivery of one or more nucleic acid vectors comprising a sequence encoding a Rep protein, and/or a protein or polypeptide of interest, into various tissues, organs, and/or cells. In some embodiments, the rAAV particle is delivered to a host cell in the presence of a Rep protein as described herein.

The wild-type AAV genome is a single-stranded deoxyribonucleic acid (ssDNA), either positive- or negative-sensed. The genome comprises two inverted terminal repeats (ITRs), one at each end of the DNA strand, two open reading frames (ORFs): rep and cap between the ITRs, and an insert nucleic acid positioned between the ITRs and optionally comprising a transgene. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

Recombinant AAV (rAAV) particles may comprise a nucleic acid vector, which may comprise at a minimum: (a) one or more transgenes comprising a sequence encoding a protein or polypeptide of interest, or one or more nucleic acid regions comprising a sequence encoding a Rep protein; and (b) one or more regions comprising inverted terminal repeat (ITR) sequences (e.g., wild-type AAV2 ITR sequences) flanking the one or more nucleic acid regions (e.g., transgenes). In some embodiments, the nucleic acid vector is between 4 kb and 5 kb in size (e.g., 4.2 to 4.7 kb in size). In some embodiments, the nucleic acid vector further comprises a region encoding a Rep protein as described herein. Any nucleic acid vector described herein may be encapsidated by a viral capsid, such as an AAV8 capsid or another serotype (e.g., a serotype that is of the same serotype as the ITR sequences), which may comprises a modified capsid protein as described herein. In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complementary vector that contains a region of the nucleic acid vector that is complementary to another region of the nucleic acid vector, initiating the formation of the double-strandedness of the nucleic acid vector.

In some embodiments, the nucleic acid vector comprises one or more transgenes comprising a sequence encoding a protein or polypeptide of interest operably linked to a promoter, wherein the one or more transgenes are flanked on each side with an ITR sequence. In some embodiments, the nucleic acid vector further comprises a region encoding a Rep protein as described herein, either contained within the region flanked by ITRs or outside the region or nucleic acid) operably linked to a promoter (e.g. an CBA promoter), wherein the one or more nucleic acid regions. The ITR sequences can be derived from any AAV serotype (e.g., 1, 2, 3, 4, 5, 6, 7, 8, 9, or 10) or can be derived from more than one serotype. In some embodiments, the ITR sequences are derived from AAV2.

ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Ca; and Addgene, Cambridge, Mass.; and Gene delivery to skeletal muscle results in sustained expression and systemic delivery of a therapeutic protein. Kessler P D, et al. Proc Natl Acad Sci USA. 1996; 93(24):14082-7; and Curtis A. Machida, Methods in Molecular Medicine™ Viral Vectors for Gene Therapy Methods and Protocols. 10.1385/1-59259-304-6:201 Humana Press Inc. 2003: Chapter 10, Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

An non-limiting example of an ITR sequence (i.e., the AAV2 ITR sequence) useful in the disclosed rAAV vectors is provided below.

### TTGGCCACTCCCTCTCTGCGCGCTCGCTCGCTCACTGAGGCCGGGCGACCAAAGG TCGCCCGACGCCCGGGCTTTGCCCGGGCGGCCTCAGTGAGCGAGCGAGCGCGCA GAGAGGGAGTGGCCAACTCCATCACTAGGGGTTCCT (SEQ ID NO: 4)

The rAAV particle, nucleic acid vector, and/or Rep protein (in any form contemplated herein) may be delivered in the form of a composition, such as a composition comprising the active ingredient, such as the rAAV particle, nucleic acid vector, and/or Rep protein (in any form contemplated herein), and a therapeutically or pharmaceutically acceptable carrier. The rAAV particles, Rep proteins, or nucleic acid vectors may be prepared in a variety of compositions, and may also be formulated in appropriate pharmaceutical vehicles for administration to human or animal subjects.

Other aspects of the disclosure are directed to methods that involve contacting cells with an rAAV preparation produced by a method described herein. The contacting may be, e.g., ex vivo or in vivo by administering the rAAV preparation to a subject. The rAAV particle or preparation may be delivered in the form of a composition, such as a composition comprising the active ingredient, such as a rAAV particle or preparation described herein, and a therapeutically or pharmaceutically acceptable excipient. The rAAV particles or preparations may be prepared in a variety of compositions and may also be formulated in appropriate pharmaceutical vehicles for administration to human or animal subjects.

In some embodiments, the nucleic acid vector comprises one or more regions comprising a sequence that facilitates expression of the nucleic acid (e.g., the transgene or the nucleic acid region encoding the Rep protein), e.g., expression control sequences operatively linked to the nucleic acid. Numerous such sequences are known in the art. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

To achieve appropriate expression levels of the protein or polypeptide of interest, any of a number of promoters suitable for use in the selected host cell may be employed. The promoter may be, for example, a constitutive promoter, tissue-specific promoter, inducible promoter, or a synthetic promoter. In arious embodiments, the promoter is a tissue-specific promoter that is active in brain cells, such as cerebral cortex cells.

Tissue-specific promoters and/or regulatory elements are also contemplated herein. In certain embodiments, the heterologous promoters of the disclosed fusion proteins are active in brain cells, such as human brain cells (e.g. human neurons and glia). Exemplary promoters active in human brain cells include, but are not limited to, the chicken beta actin (CBA) promoter, the cytomegalovirus (CMV) promoter, the transthyretin promoter, the desmin promoter, the PGK promoter, the GAD67 promoter, the Dlx5/6 promoter, the Drd1a promoter, the human synapsin 1 gene promoter (SYN), the hybrid CMV enhancer/chicken β-actin (CAG) promoter, Glial Fibrillary Acidic protein (GFAP) promoter, Microtubule-associated protein 2 (MAP2) promoter, and the platelet-derived growth factor-0 chain promoter (1500 bp). These promoters are described in, e.g., Kugler et al. Virology, 311(1):89-95 (2003) and Morelli et al. J Gen Virol., 80 (Pt 3):571-83 (1999), which are incorporated herein by reference. Non-limiting examples of such promoters that may be used include species-specific promoters, such as human-specific promoters.

Other regulatory elements may also be contemplated for achieving appropriate expression levels of the heterologous nucleic acid. For examples, any of the disclosed rAAV vector may comprise a Woodchuck Hepatitis VirusPosttranscriptional Regulatory element (WPRE), e.g., a WPREsf element. A WPRE is a DNA sequence that, when transcribed, forms a tertiary structure that enhances expression of the vector.

The rAAV particle or particle within an rAAV preparation may be of any AAV serotype, including any derivative or pseudotype (e.g., 1, 2, 3, 4, 5, 6, 7, 8, 9, 10, 2/1, 2/5, 2/8, 2/9, 3/1, 3/5, 3/8, or 3/9). As used herein, the serotype of an rAAV particle refers to the serotype of the capsid proteins of the recombinant virus. In some embodiments, the capsid protein of the rAAV particle is AAV8, or a variant thereof. In other embodiments, the capsid protein of the rAAV particle is AAV1, AAV2, AAV3, AAV5, AAV6, AAV8, AAV9, AAV10, or a variant thereof.

In some embodiments, the capsid protein is a variant, derivative or pseudotype of a wild-type protein. Non-limiting examples of derivatives and pseudotypes include rAAV2/8, rAAV2/1, rAAV2/5, rAAV2/9, AAV2-AAV3 hybrid, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, AAV7BP2, AAVphp.B, and AAVr3.45. Such AAV serotypes and derivatives/pseudotypes, and methods of producing such derivatives/pseudotypes are known in the art (see, e.g., Asokan A1, Schaffer DV, Samulski RJ, The AAV vector toolkit: poised at the clinical crossroads, Mol Ther. 2012; 20(4):699-708). In some embodiments, the rAAV particle is a pseudotyped rAAV particle, which comprises (a) a nucleic acid vector comprising ITRs from one serotype (e.g., AAV2, AAV3) and (b) a capsid comprised of capsid proteins derived from another serotype (e.g., AAV1, AAV2, AAV3, AAV4, AAV5, AAV6, AAV7, AAV8, AAV9, or AAV10). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001). In some embodiments, the rAAV particle comprises a capsid that includes modified capsid proteins (e.g., capsid proteins comprising a modified VP3 region). Methods of producing modified capsid proteins are known in the art (see, e.g., US Patent Publication No. 2013/0310443, incorporated herein by reference in its entirety). An rAAV9 vector comprising NAGLU operably controlled by a cytomegalovirus promoter recently entered clinical trials for treatment of MPS IIIB (see Clinical Trial NCT03315182).

In some embodiments, the rAAV particle comprises a heterologous nucleic acid sequence, encoding a therapeutic or diagnostic agent, e.g., a codon-optimized NAGLU. The heterologous nucleic acid sequence may be in the form of a single-stranded (ss) or self-complementary (sc) AAV nucleic acid vector.

Accordingly, in some aspects, the disclosure provides an rAAV particle comprising a capsid comprising a VP1, VP2, and/or VP3 protein, wherein the rAAV particle further comprises a polynucleotide comprising a heterologous nucleic acid sequence. In some embodiments, the rAAV particle comprises a capsid comprising a VP1, VP2, and/or VP3 protein, wherein the VP1 protein comprises the amino acid sequence of SEQ ID NO: 3.

In some embodiments, the disclosure provides a capsid protein comprising an amino acid sequence having at least 80% identity, at least 85% identity, at least 90% identity, at least 92.5% identity, at least 95% identity, 98% identity, or 99% identity to any of SEQ ID NO: 3. In some embodiments, the disclosure provides a capsid protein comprising the amino acid sequence of SEQ ID NO: 3. In some embodiments, the disclosed capsids may comprise a sequence having 1, 2, 3, 4, 5, 6, 7, 8, 9, 10, 11, 12, 13, 14, 15, 16, 17, or 18 amino acids that differ relative to the sequence of SEQ ID NO: 3. These differences may comprise amino acids inserted, deleted, or substituted relative to the sequence of SEQ ID NO: 3.

### Production Methods

Methods of producing rAAV particles and nucleic acid vectors are described herein. Other methods are also known in the art and commercially available (see, e.g., Zolotukhin et al. Production and purification of serotype 1, 2, and 5 recombinant adeno-associated viral vectors. Methods 28 (2002) 158-167; and U.S. Patent Publication Nos. US 2007/0015238 and US 2012/0322861, which are incorporated herein by reference; and plasmids and kits available from ATCC and Cell Biolabs, Inc.). For example, a plasmid containing the nucleic acid vector (i.e., a replicating plasmid) may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified. In some embodiments, the producer cell line is human HEK293 cells.

In some embodiments, the one or more helper plasmids include a first helper plasmid comprising a rep gene and a cap gene and a second helper plasmid comprising a Ela gene, a E1b gene, a E4 gene, a E2a gene, and a VA gene. Exemplary AAV Rep protein sequences are provided herein. In some embodiments, the rep gene is a rep gene derived from AAV2 or AAV8 and the cap gene is derived from AAV2 or AAV8 and may include modifications to the gene in order to produce the modified capsid protein described herein. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Ca; and Addgene, Cambridge, Mass.; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081.; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188). Exemplary rAAV purification methods are described in U.S. Pat. No. 10,526,583, herein incorporated by reference.

In some embodiments, a replicating plasmid is used to encode the rAAV nucleic acid sequence comprising coNAGLU flanked by ITRs (e.g., the genome of SEQ ID NO: 2), enable replication in a bacterial host, and enable packaging in mammalian host cells, e.g. HEK293 cells. In some embodiments, the replicating plasmid is transfected into HEK293 cells for packaging. When transfected into host cells in combination with a pHelper plasmid and/or a Rep-Cap plasmid, the replicating plasmid will generate AAV-tcm8 viral particles that will cause transduced cells to produce coNAGLU.

In some embodiments, the replicating plasmid is a plasmid having at least 90% identity, at least 95% identity, at least 98% identity, or at least 99% identity to the nucleotide sequence of SEQ ID NO: 5. In some embodiments, the plasmid comprises the nucleotide sequence of SEQ ID NO: 5. This sequence is about 6,840 nucleotides in length and is provided below.

(SEQ ID NO: 5)
GGGGGGGGGGGGGGGGGGTTGGCCACTCCCTCTCT
GCGCGCTCGCTCGCTCACTGAGGCCGGGCGACCAA
AGGTCGCCCGACGCCCGGGCTTTGCCCGGGCGGCC
TCAGTGAGCGAGCGAGCGCGCAGAGAGGGAGTGGC
CAACTCCATCACTAGGGGTTCCTAGATCTGAATTC
GGTACCCTAGTTATTAATAGTAATCAATTACGGGG
TCATTAGTTCATAGCCCATATATGGAGTTCCGCGT
TACATAACTTACGGTAAATGGCCCGCCTGGCTGAC
CGCCCAACGACCCCCGCCCATTGACGTCAATAATG
ACGTATGTTCCCATAGTAACGCCAATAGGGACTTT
CCATTGACGTCAATGGGTGGACTATTTACGGTAAA
CTGCCCACTTGGCAGTACATCAAGTGTATCATATG
CCAAGTACGCCCCCTATTGACGTCAATGACGGTAA
ATGGCCCGCCTGGCATTATGCCCAGTACATGACCT
TATGGGACTTTCCTACTTGGCAGTACATCTACGTA
TTAGTCATCGCTATTACCATGGTCGAGGTGAGCCC
CACGTTCTGCTTCACTCTCCCCATCTCCCCCCCCT
CCCCACCCCCAATTTTGTATTTATTTATTTTTTAA
TTATTTTGTGCAGCGATGGGGGCGGGGGGGGGGGG
GGGGCGCGCGCCAGGCGGGGCGGGGCGGGGCGAGG
GGCGGGGCGGGGCGAGGCGGAGAGGTGCGGCGGCA
GCCAATCAGAGCGGCGCGCTCCGAAAGTTTCCTTT
TATGGCGAGGCGGCGGCGGCGGCGGCCCTATAAAA
AGCGAAGCGCGCGGCGGGCGGGAGTCGCTGCGACG
CTGCCTTCGCCCCGTGCCCCGCTCCGCCGCCGCCT
CGCGCCGCCCGCCCCGGCTCTGACTGACCGCGTTA
CTCCCACAGGTGAGCGGGCGGGACGGCCCTTCTCC
TCCGGGCTGTAATTAGCGCTTGGTTTAATGACGGC
TTGTTTCTTTTCTGTGGCTGCGTGAAAGCCTTGAG
GGGCTCCGGGAGCTAGAGCCTCTGCTAACCATGTT
CATGCCTTCTTCTTTTTCCTACAGCTCCTGGGCAA
CGTGCTGGTTATTGTGCTGTCTCATCATTTTGGCA
AAGAATTCATGGAGGCTGTGGCTGTGGCTGCCGCT
GTGGGAGTGCTGCTGCTGGCTGGGGCCGGAGGGGC
CGCTGGCGATGAGGCTCGGGAGGCTGCAGCTGTGC
GCGCACTGGTCGCACGACTGCTGGGACCTGGGCCA
GCAGCCGACTTCTCTGTGAGTGTCGAGAGAGCCCT
GGCTGCAAAGCCCGGACTGGATACCTACAGTCTGG
GAGGAGGAGGAGCAGCTCGAGTGAGGGTCAGAGGG
TCAACAGGAGTGGCAGCAGCTGCAGGACTGCACCG
ATATCTGCGAGACTTTTGCGGCTGTCATGTGGCCT
GGTCAGGAAGCCAGCTGAGGCTGCCCAGACCTCTG
CCAGCAGTGCCAGGCGAGCTGACAGAAGCCACTCC
CAACCGGTACCGCTACTATCAGAACGTGTGCACCC
AGTCCTATTCTTTCGTCTGGTGGGACTGGGCTCGA
TGGGAGCGCGAAATCGATTGGATGGCACTGAACGG
AATTAATCTGGCACTGGCATGGTCCGGACAGGAGG
CAATCTGGCAGAGAGTGTACCTGGCACTGGGACTG
ACTCAGGCCGAGATTAACGAGTTCTTCACCGGGCC
AGCTTTTCTGGCATGGGGACGGATGGGGAATCTGC
ACACATGGGACGGACCACTGCCACCTTCTTGGCAC
ATCAAACAGCTGTATCTGCAGCATAGGGTGCTGGA
TCAGATGAGAAGTTTTGGCATGACTCCAGTGCTGC
CCGCTTTCGCAGGACACGTCCCTGAGGCCGTGACA
CGCGTCTTCCCACAGGTGAACGTCACTAAGATGGG
CAGCTGGGGACACTTCAACTGCAGTTACTCATGTA
GCTTCCTGCTGGCCCCTGAAGATCCAATTTTTCCC
ATCATTGGAAGCCTGTTCCTGCGGGAGCTGATCAA
AGAATTTGGAACCGACCACATCTACGGGGCCGATA
CATTCAACGAGATGCAGCCACCCAGCTCCGAACCT
AGCTACCTGGCCGCTGCAACCACAGCCGTGTACGA
GGCCATGACCGCTGTGGACACAGAAGCCGTCTGGC
TGCTGCAGGGGTGGCTGTTTCAGCATCAGCCACAG
TTCTGGGGACCTGCACAGATCCGAGCTGTGCTGGG
AGCAGTCCCACGAGGAAGGCTGCTGGTGCTGGATC
TGTTCGCTGAGTCCCAGCCCGTCTACACTAGGACC
GCCTCTTTCCAGGGCCAGCCTTTTATTTGGTGTAT
GCTGCACAACTTTGGAGGGAATCATGGGCTGTTCG
GCGCACTGGAGGCAGTGAACGGAGGACCAGAAGCA
GCTAGACTGTTTCCTAATAGCACTATGGTGGGCAC
CGGAATGGCTCCCGAGGGCATCTCACAGAATGAAG
TGGTCTACAGCCTGATGGCAGAGCTGGGATGGCGA
AAGGACCCCGTGCCTGATCTGGCAGCCTGGGTCAC
TAGTTTCGCTGCAAGGAGATACGGGGTGTCACACC
CTGACGCTGGAGCAGCTTGGCGACTGCTGCTGAGG
AGCGTGTACAACTGCAGTGGGGAGGCCTGTAGAGG
CCATAATCGGTCCCCACTGGTGCGGCGCCCCTCAC
TGCAGATGAACACCAGCATCTGGTACAATCGATCC
GACGTGTTCGAAGCTTGGCGGCTGCTGCTGACAAG
TGCCCCTTCACTGGCTACTTCTCCAGCATTCCGCT
ATGACCTGCTGGATCTGACAAGGCAGGCCGTGCAG
GAGCTGGTCAGCCTGTACTATGAGGAAGCTCGCAG
CGCATACCTGTCCAAGGAACTGGCATCCCTGCTGA
GGGCAGGAGGCGTGCTGGCTTATGAGCTGCTGCCA
GCTCTGGACGAAGTCCTGGCATCCGATTCTAGATT
TCTGCTGGGCAGCTGGCTGGAGCAGGCACGAGCAG
CAGCCGTGAGCGAGGCCGAAGCTGACTTCTACGAG
CAGAACTCTAGGTATCAGCTGACTCTGTGGGGACC
CGAAGGGAACATCCTGGATTACGCAAACAAGCAGC
TGGCAGGACTGGTGGCTAATTACTATACCCCTAGA
TGGCGGCTGTTTCTGGAGGCCCTGGTGGACTCTGT
CGCTCAGGGCATTCCATTCCAGCAGCACCAGTTTG
ATAAGAACGTGTTCCAGCTGGAACAGGCCTTCGTC
CTGTCTAAACAGCGGTATCCTAGTCAGCCACGCGG
CGACACAGTGGACCTGGCAAAGAAGATTTTCCTGA
AATACTATCCCCGCTGGGTGGCTGGCTCATGGACC
GGTGGCGCGCCTTCGAACTGCAGCTCGAGGTAGGA
TCCAGACATGATAAGATACATTGATGAGTTTGGAC
AAACCACAACTAGAATGCAGTGAAAAAAATGCTTT
ATTTGTGAAATTTGTGATGCTATTGCTTTATTTGT
AACCATTATAAGCTGCAATAAACAAGTTAACAACA
ACAATTGCATTCATTTTATGTTTCAGGTTCAGGGG
GAGGTGTGGGAGGTTTTTTAGTCGACTAGAGCTCG
CTGATCAGCCTCGACTGTGCCTTCTAGTTGCCAGC
CATCTGTTGTTTGCCCCTCCCCCGTGCCTTCCTTG
ACCCTGGAAGGTGCCACTCCCACTGTCCTTTCCTA
ATAAAATGAGGAAATTGCATCGCATTGTCTGAGTA
GGTGTCATTCTATTCTGGGGGGTGGGGTGGGGCAG
GACAGCAAGGGGGAGGATTGGGAAGACAATAGCAG
GCATGCTGGGGAGAGATCTAGGAACCCCTAGTGAT
GGAGTTGGCCACTCCCTCTCTGCGCGCTCGCTCGC
TCACTGAGGCCGCCCGGGCAAAGCCCGGGCGTCGG
GCGACCTTTGGTCGCCCGGCCTCAGTGAGCGAGCG
AGCGCGCAGAGAGGGAGTGGCCAACCCCCCCCCCC
CCCCCCCTGCAGCCAGCTGGCGTAATAGCGAAGAG
GCCCGCACCGATCGCCCTTCCCAACAGTTGCGTAG
CCTGAATGGCGAATGGCGCGACGCGCCCTGTAGCG
GCGCATTAAGCGCGGCGGGTGTGGTGGTTACGCGC
AGCGTGACCGCTACACTTGCCAGCGCCCTAGCGCC
CGCTCCTTTCGCTTTCTTCCCTTCCTTTCTCGCCA
CGTTCGCCGGCTTTCCCCGTCAAGCTCTAAATCGG
GGGCTCCCTTTAGGGTTCCGATTTAGTGCTTTACG
GCACCTCGACCCCAAAAAACTTGATTAGGGTGATG
GTTCACGTAGTGGGCCATCGCCCTGATAGACGGTT
TTTCGCCCTTTGACGTTGGAGTCCACGTTCTTTAA
TAGTGGACTCTTGTTCCAAACTGGAACAACACTCA
ACCCTATCTCGGTCTATTCTTTTGATTTATAAGGG
ATTTTGCCGATTTCGGCCTATTGGTTAAAAAATGA
GCTGATTTAACAAAAATTTAACGCGAATTTTAACA
AAATATTAACGTTTACAATTTCCTGATGCGGTATT
TTCTCCTTACGCATCTGTGCGGTATTTCACACCGC
ATATGGTGCACTCTCAGTACAATCTGCTCTGATGC
CGCATAGTTAAGCCAGCCCCGACACCCGCCAACAC
CCGCTGACGCGCCCTGACGGGCTTGTCTGCTCCCG
GCATCCGCTTACAGACAAGCTGTGACCGTCTCCGG
GAGCTGCATGTGTCAGAGGTTTTCACCGTCATCAC
CGAAACGCGCGAGACGAAAGGGCCTCGTGATACGC
CTATTTTTATAGGTTAATGTCATGATAATAATGGT
TTCTTAGACGTCAGGTGGCACTTTTCGGGGAAATG
TGCGCGGAACCCCTATTTGTTTATTTTTCTAAATA
CATTCAAATATGTATCCGCTCATGAGACAATAACC
CTGATAAATGCTTCAATAATATTGAAAAAGGAAGA
GTATGAGTATTCAACATTTCCGTGTCGCCCTTATT
CCCTTTTTTGCGGCATTTTGCCTTCCTGTTTTTGC
TCACCCAGAAACGCTGGTGAAAGTAAAAGATGCTG
AAGATCAGTTGGGTGCACGAGTGGGTTACATCGAA
CTGGATCTCAACAGCGGTAAGATCCTTGAGAGTTT
TCGCCCCGAAGAACGTTTTCCAATGATGAGCACTT
TTAAAGTTCTGCTATGTGGCGCGGTATTATCCCGT
ATTGACGCCGGGCAAGAGCAACTCGGTCGCCGCAT
ACACTATTCTCAGAATGACTTGGTTGAGTACTCAC
CAGTCACAGAAAAGCATCTTACGGATGGCATGACA
GTAAGAGAATTATGCAGTGCTGCCATAACCATGAG
TGATAACACTGCGGCCAACTTACTTCTGACAACGA
TCGGAGGACCGAAGGAGCTAACCGCTTTTTTGCAC
AACATGGGGGATCATGTAACTCGCCTTGATCGTTG
GGAACCGGAGCTGAATGAAGCCATACCAAACGACG
AGCGTGACACCACGATGCCTGTAGCAATGGCAACA
ACGTTGCGCAAACTATTAACTGGCGAACTACTTAC
TCTAGCTTCCCGGCAACAATTAATAGACTGGATGG
AGGCGGATAAAGTTGCAGGACCACTTCTGCGCTCG
GCCCTTCCGGCTGGCTGGTTTATTGCTGATAAATC
TGGAGCCGGTGAGCGTGGGTCTCGCGGTATCATTG
CAGCACTGGGGCCAGATGGTAAGCCCTCCCGTATC
GTAGTTATCTACACGACGGGGAGTCAGGCAACTAT
GGATGAACGAAATAGACAGATCGCTGAGATAGGTG
CCTCACTGATTAAGCATTGGTAACTGTCAGACCAA
GTTTACTCATATATACTTTAGATTGATTTAAAACT
TCATTTTTAATTTAAAAGGATCTAGGTGAAGATCC
TTTTTGATAATCTCATGACCAAAATCCCTTAACGT
GAGTTTTCGTTCCACTGAGCGTCAGACCCCGTAGA
AAAGATCAAAGGATCTTCTTGAGATCCTTTTTTTC
TGCGCGTAATCTGCTGCTTGCAAACAAAAAAACCA
CCGCTACCAGCGGTGGTTTGTTTGCCGGATCAAGA
GCTACCAACTCTTTTTCCGAAGGTAACTGGCTTCA
GCAGAGCGCAGATACCAAATACTGTCCTTCTAGTG
TAGCCGTAGTTAGGCCACCACTTCAAGAACTCTGT
AGCACCGCCTACATACCTCGCTCTGCTAATCCTGT
TACCAGTGGCTGCTGCCAGTGGCGATAAGTCGTGT
CTTACCGGGTTGGACTCAAGACGATAGTTACCGGA
TAAGGCGCAGCGGTCGGGCTGAACGGGGGGTTCGT
GCACACAGCCCAGCTTGGAGCGAACGACCTACACC
GAACTGAGATACCTACAGCGTGAGCATTGAGAAAG
CGCCACGCTTCCCGAAGGGAGAAAGGCGGACAGGT
ATCCGGTAAGCGGCAGGGTCGGAACAGGAGAGCGC
ACGAGGGAGCTTCCAGGGGGAAACGCCTGGTATCT
TTATAGTCCTGTCGGGTTTCGCCACCTCTGACTTG
AGCGTCGATTTTTGTGATGCTCGTCAGGGGGGCGG
AGCCTATGGAAAAACGCCAGCAACGCGGCCTTTTT
ACGGTTCCTGGCCTTTTGCTGGCCTTTTGCTCACA
TGTTCTTTCCTGCGTTATCCCCTGATTCTGTGGAT
AACCGTATTACCGCCTTTGAGTGAGCTGATACCGC
TCGCCGCAGCCGAACGACCGAGCGCAGCGAGTCAG
TGAGCGAGGAAGCGGAAGAGCGCCCAATACGCAAA
CCGCCTCTCCCCGCGCGTTGGCCGATTCATTAATG
CAGCTGGGCTGCA

A non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. In some embodiments, the one or more helper plasmids comprise rep genes for a first serotype (e.g., AAV3, AAV5, and AAV6), cap genes (which may or may not be of the first serotype) and optionally one or more of the adenoviral VA, E2A (DBP), and E4 genes under the transcriptional control of their native promoters. In some embodiments, the one or more helper plasmids comprise cap ORFs (and optionally rep ORFs) for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO₄-mediated transfection, lipids or polymeric molecules such as Polyethylenimine (PEI) with the helper plasmid(s) and a plasmid containing a nucleic acid vector described herein. The HEK293 cells are then incubated for at least 60 hours to allow for rAAV particle production. Alternatively, in another example Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known the art or described herein, e.g., by iodixanol step gradient, CsCl gradient, chromatography, or polyethylene glycol (PEG) precipitation.

In various embodiments, an iodixanol step gradient purification method is used. Vectors may be packaged into mammalian cells (e.g., HEK293T cells) and purified by iodixanol gradient centrifugation, followed by buffer exchange and concentration into BSS/Tween buffer. An affinity capture step may be added.

### Subjects

Aspects of the disclosure relate to methods and preparations for use with a subject, such as human or non-human primate subjects, a host cell in situ in a subject, or a host cell derived from a subject. In various embodiments, the subject is human.

In some embodiments, the subject is not a human. In some embodiments, the subject is a domesticated animal. In some embodiments, the subject is an experimental animal. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other exemplary subjects include domesticated animals such as dogs and cats; livestock such as horses, cattle, pigs, sheep, goats, and chickens; and other animals such as rodents (mice, rats, guinea pigs, and hamsters).

In some embodiments, the subject has or is suspected of having a disease that may be treated with gene therapy. In some embodiments, the subject has or is suspected of having Sanfilippo syndrome B or MPS IIIB. In particular embodiments, the subject has or is suspected of having Sanfilippo syndrome B or MPS IIIB.

### Examples

### Example 1

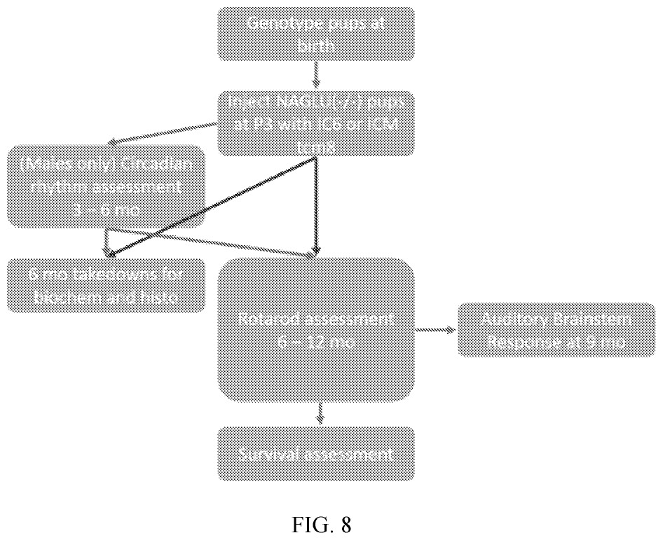

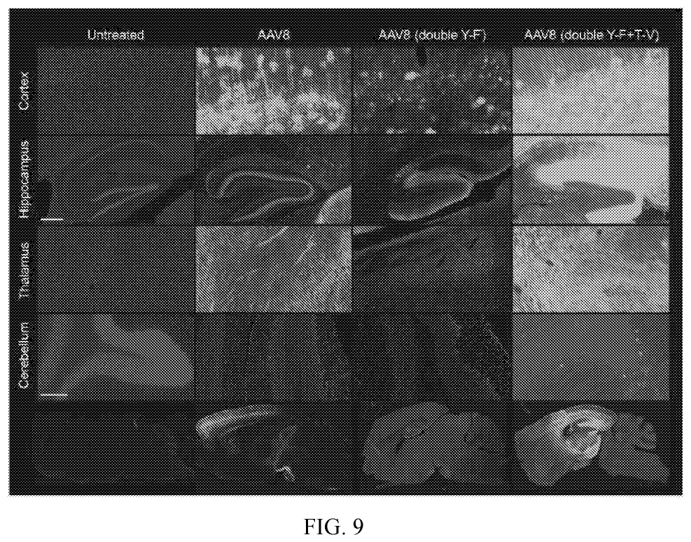

NAGLU^{-/-} mice, an MPS IIIB murine model, were used for the experiments disclosed herein. A schematic overview of the experiments is shown in FIG. 8. NAGLU^{-/-} mice were administered AAVtcm8 vector expressing codon-optimized NAGLU (AAVtcm8-coNAGLU) three days after birth (post-natal 3, or P3) by intraparenchymal 6-site injection into bilateral frontal cortex, temporal cortex and cerebellum (IC6) or cisternal magna injection (ICM) at a total dose of 1.8×10¹⁰ vgs/ml. Mice in the wild-type or heterozygous littermate group (a control group herein referred to as the “normal” group), untreated MPS IIIB control, IC6⁺ Naglu^{-/-} (IC6) and ICM⁺Naglu^{-/-} (ICM) groups were monitored for three months for survival and behavioral responses. Subsequently, the neurological activity of 4-5 month old male mice were assessed using a running wheel. At 6 months, both male and females underwent rotarod performance tests. Likewise, both male and female mice of between 8 and 9 months of age in the study underwent Auditory-evoked Brainstem Response (ABR) assessments to measure hearing acuity. Histological examination of lysosomal distention and NAGLU activity were assessed on brains of animals from each group at 6 months of age.

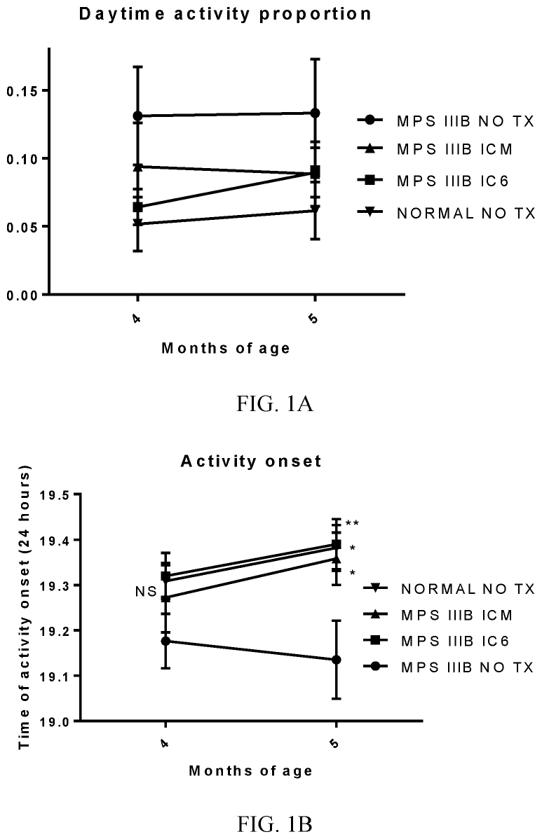

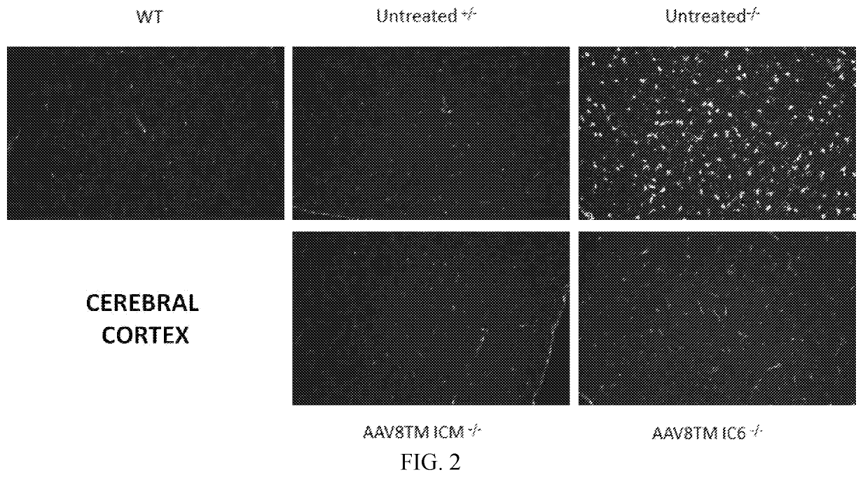

As shown in FIGS. 1A and 1B, male mice (N=8-10 per group) were exposed to 12-hour light/dark cycles, and their activity was recorded using the Clocklab software. A proportion of total activity performed while ambient lights were on shows a trend toward more daytime activity in untreated MPS IIIB mice with improvements in both treated groups (FIG. 1A). Activity onset time in 20 week old mice with lights off is shown in FIG. 1B. The measured activities in each of the four mice groups did not significantly differ.

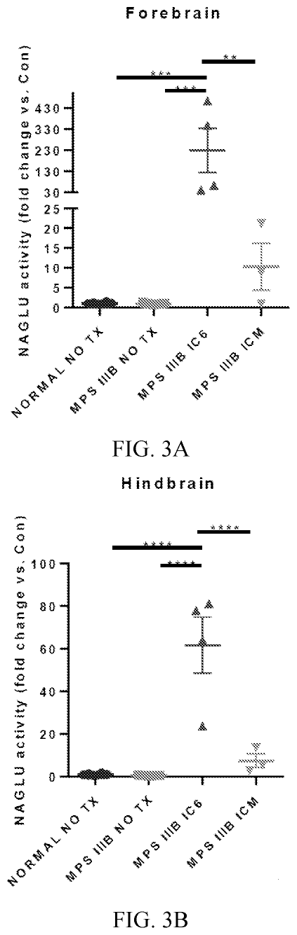

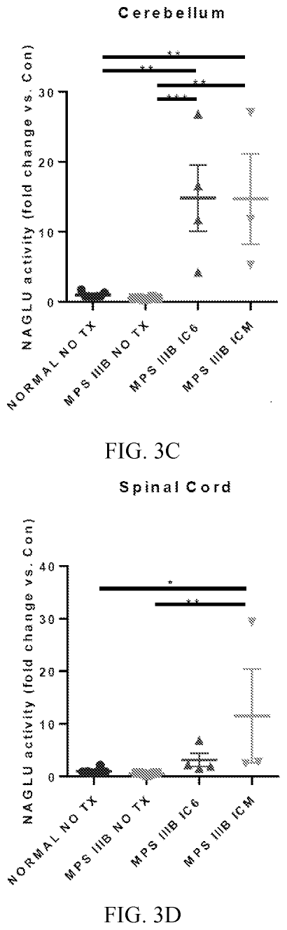

Administration of AAVtcm8-coNAGLU by IC6 or ICM injection resulted in elevation of NAGLU enzymatic activity and improvement in histologic indices of disease. NAGLU activity was enhanced many-fold in all evaluated regions of the CNS. Brain and spinal cord sections from 6 month old mice from each group were flash frozen and lysed. NAGLU expression was measured by 4-methyl-umbelliferone (4MU) fluorescence intensity assay. As shown in FIGS. 3A-3D, compared to normal group, an enhancement in vector-mediated NAGLU expression was observed into the forebrain after vector administration by IC6 (227-fold greater expression) and by ICM (9-fold greater expression); and into the hindbrain after vector administration by IC6 (62-fold greater) and by ICM (7-fold greater) Vector administration into the cerebellum by IC6 and ICM showed 15-fold greater expression than normal, and administration into the spinal cord by IC6 showed 3-fold greater and by ICM showed 12-fold greater expression.

Antibody staining of the cerebral cortex for the Lysosomal-associated membrane protein 1 (LAMP) shows that lysosomal storage deficiencies in MPS IIIB mice were corrected by administration of AAVtcm8-coNAGLU. LAMP staining data suggests the degree of lysosomal distention, a clinically relevant phenotype in MPS IIIB. Brains from six-month old mice in each of the four groups were fixed and immunohisto-chemistry for LAMP was performed. As shown in FIG. 2, significant reduction in lysosomal distention was observed in mice receiving IC6 or ICM when compared to Naglu^{-/-} LAMP staining area percentage. Specifically, LAMP staining data in the cortex for each group is as follows: Normal, 0.1 [p<0.0001 vs. untreated]; Naglu^{-/-} untreated, 3.6; IC6, 0.3 [p<0.0001 vs. untreated]; and ICM, 0.2 [p<0.0001 vs. untreated]. LAMP staining data in the cerebellum for each group is as follows: Normal, 0.2 [p<0.0001 vs. untreated]; Naglu^{-/-} untreated, 2.2; IC6, 0.6 [p=0.0002 vs. untreated]; and ICM, 0.3 [p<0.0001 vs. untreated]).

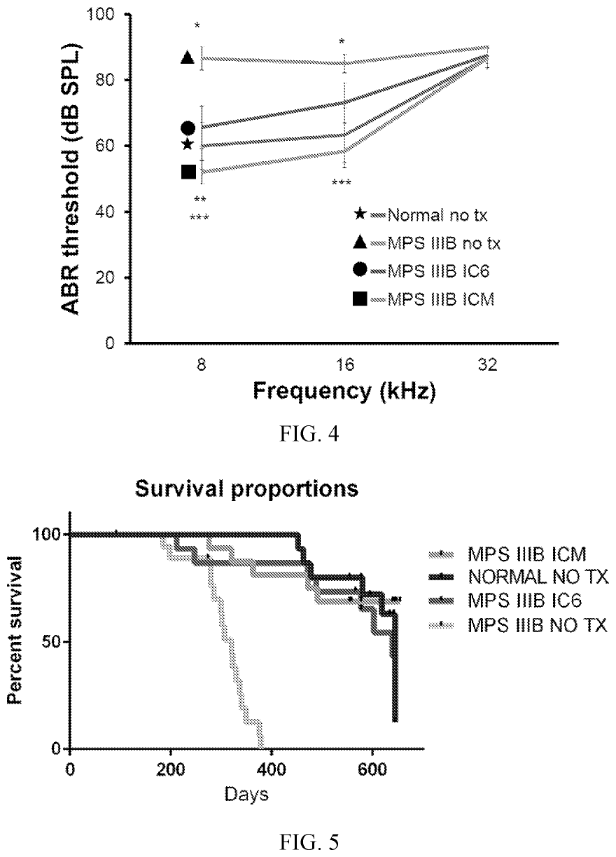

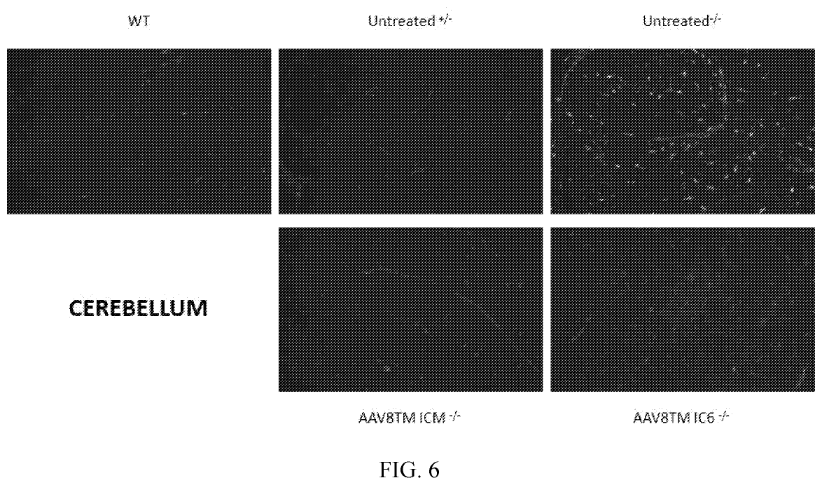

ABR thresholds were improved in mice following vector administration. ABR data was measured using tone burst stimuli at 8, 16, and 32 kHz in Normal (“Norm”) untreated mice, NAGLU^{-/-} (“Mut”) untreated mice, Mut IC6 mice, and Mut ICM mice at 9 months of age (N=8-15). As shown in FIG. 4, a significant improvement of hearing deficits was observed at the 8 kHz and 16 kHz tone burst frequencies. At the 8 kHz frequency, the following thresholds were observed: Normal group: 60 decibels of sound pressure level (dB SPL) [p<0.001 vs untreated]; Naglu^{-/-} untreated group: 87 dB SPL; IC6: 66 dB SPL [p<0.05 vs untreated]; and ICM: 52 dB SPL [p<0.0001 vs untreated]). At the 16 kHz frequency, the following thresholds were observed: Normal: 63 dB SPL [p<0.01 vs untreated]; Naglu^{-/-} untreated: 85 dB SPL; IC6: 73 dB SPL [p=0.4631 vs untreated]; and ICM: 58 dB SPL [p<0.001 vs untreated]).

A very important finding is that the administration of the AAVtcm8-coNAGLU vector prolongs survival in MPS IIIB mice to near-wild type levels. The MPS IIIB untreated group's median survival was 320 days, and both treatment groups had significantly prolonged survival that was not different from the Normal group at 580 days post-vector administration. As shown in FIG. 5, an extension of median lifespan was observed in IC6 and ICM animals compared to Naglu^{-/-} Normal 644 days [p<0.0001 vs untreated] and Naglu^{-/-} untreated, which had a median survival of 320 days. The median survival for the IC6-treated group was 639 days at [p<0.0001 vs untreated], and for the ICM-treated group was not reached [p<0.0001 vs untreated]).

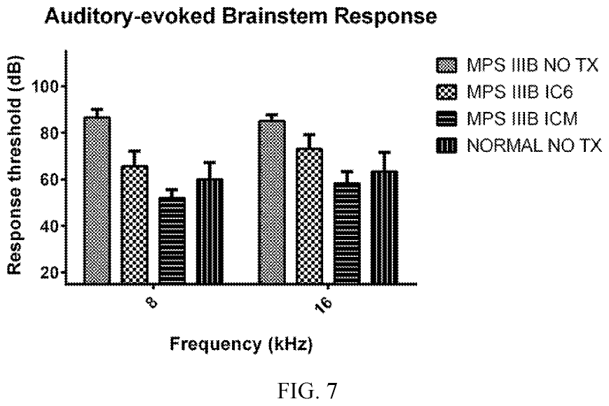

As shown in FIGS. 6 and 7, mice injected by either method experienced complete or near complete restoration of hearing as measured by auditory evoked brainstem responses at 8-9 months of age and complete correction of lysosomal storage deficits in the cortex and cerebellum by immunohistochemical staining of lysosomes using antibodies to Lysosomal Associated Membrane Protein (LAMP-1). Antibody staining of Lysosomal-associated membrane protein 1 (LAMP) in the cerebellum (FIG. 6) shows similar size and distribution of staining in the treated groups to that seen in unaffected normal animals rather than the large lysosomes seen in untreated MPS IIIB mouse brains. Auditory-evoked brainstem response (ABR) of the four groups of mice at 8 and 16 kHz illustrate that the volume threshold needed to detect a brainstem response was >85 decibels for untreated MPS IIIB animals (above range of speaker) and 60/63 decibels in untreated normal animals (p<0.01/<0.05 vs MPS IIIB no treatment), 52/58 for ICM treated animals (p<0.0001/<0.001 vs MPS IIIB no treatment and NS vs normal) and 65/73 for IC6 treated animals (p<0.05/NS vs MPS IIIB no treatment and NS vs normal).

In conclusion, both intraparenchymal and intracisternal routes of administration of the AAVtcm8-coNAGLU vector resulted in improvement in all evaluations of NAGLU-associated function in which Normal untreated mice differed from MPS IIIB untreated mice. Comparing the two routes of administration, the IC6 injection route confers higher brain NAGLU activity than ICM injection. At the same time, the ICM injection route completely restored auditory function in evaluated mice.

These results demonstrate that neonatal treatment of MPS IIIB mice with AAVtcm8-coNAGLU either by IC6 or ICM injections resulted in above normal NAGLU activity and normalization of lysosomal storage, hearing and survival. In addition, lysosomal storage function in the brain is normalized following administration of the AAVtcm8-coNAGLU vector by the ICM or IC6 routes. Improvement of hearing and lysosomal storage in the mice subjects were demonstrated for a period of 1.5 years. This data suggests that AAVtcm8-coNAGLU could be used clinically for restoration of hearing defects in MPS IIIB subjects for a period of 1 year, 1.5 years, or longer.

### Example 2: Transduction Assessment of AAV8 Double and Triple Mutants in Brain Tissues

### Introduction

One critical component of effective brain delivery is widespread delivery of therapeutic products throughout the central nervous system (CNS), as most of these disorders diffusely affect the CNS [1, 2]. Therefore, identifying a gene transfer injection method and vector that will diffuse through the brain more effectively is critical for any disorder that results from somatic mutations that are ubiquitous. The gene therapy results obtained in models of an array of autosomal recessive lysosomal disorders that predominantly affect the brain indicate that more widespread transduction and protein expression will result in better therapeutic effects in vivo. In addition, identifying the most efficient transduction regimen should also result in a safer therapeutic strategy. The requirement for efficient viral transduction is especially true of lysosomal disorders whose most devastating symptoms are the result of widespread brain pathology. Given that currently available recombinant adeno-associated viral vectors (rAAV) do not globally transduce the brain after peripheral injection, there are two basic parameters that can be modified to improve distribution of brain transduction: improving the efficiency of vector transduction via capsid alterations and/or choosing the most efficient route of injection. Recent studies, aimed at improving understanding of the cellular roadblocks affecting the efficiency of adeno-associated viral (AAV) transduction, revealed that the ubiquitin-proteasome pathway plays an essential role in AAV2 intracellular trafficking [3,4,5]. This is mediated at least in part by epidermal growth factor receptor protein tyrosine kinase (EGFR-PTK). Additionally, a host cell protein, FK506-binding protein 52 (FKBP52) in its tyrosine-phosphorylated form, prevents viral second-strand DNA synthesis, resulting in inhibition of AAV-mediated transgene expression [6]. Both the viral capsid and human FKBP52 protein can be phosphorylated by EGFR-PTK [7]. This effect results in substantial numbers of ubiquitinated virions being recognized and targeted for proteosomal degradation on their way to the nucleus, and inefficient second-strand synthesis, thus leading to inefficient nuclear transport. Phosphorylation prior to ubiquitination can occur at tyrosine, serine, or threonine residues. Therefore, substitution of surface-exposed tyrosine or threonine residues on AAV2 capsids was undertaken to allow the vectors to escape ubiquitination and subsequent degradation. Importantly, mutagenesis of highly conserved exposed tyrosine residues (Y444F, Y500F, or Y730F) on AAV2 capsids enhanced transduction up to tenfold in HeLa cells and 30-fold in mouse liver [8]. Since then, single or combined tyrosine mutants of AAV2 have been successfully tested in vitro in fibroblasts and mesenchymal stem cells [9] and in vivo in murine hepatocytes [10], and the retina [3]. Improved transduction of mouse skeletal muscle was also obtained with tyrosine mutants of AAV8 in the lungs [11] and in the skeletal muscle by AAV6 vectors [12]. In our previous studies, neonatal intracranial administration of AAV5, AAV8, AAV9, and AAVrh10 was compared, and AAV8 showed superiority to AAV5, AAV9, and AAVrh10 in its ability to foster robust and widespread transduction within the mucopolysaccharidosis type IIIB (MPS IIIB) brain [13, 14]. Consistent with previous studies, AAV8 expressed a preference for neurons and astrocytes when injected in neonates. To improve upon the therapeutic capacity of AAV8, the improved transduction efficiency of modified capsid vectors in somatic organs was further evaluated. The use of AAV8 capsid tyrosine mutants in the brain of MPS IIIB animals has thus far not been assessed. In particular, based on its efficiency in a retinal model, AAV8(double Y-F) ((AAV8(Y447F+Y733F)) and AAV8(double Y-F+T-V) (AAV8(Y447F+Y733F+T494V)) were selected for further analysis.

To address the variable of the most efficient injection route to transduce the CNS, the aforementioned modified rAAV8 vectors were administered either via an intraparenchymal six site (IC6) route, an intracerebroventricular (ICV) injection, intrathalamic (TH), and ventral tegmental area (VTA) methods. As the six-site method, which had been tested previously, is more invasive, requiring six burr holes, the goal is to identify an alternate less invasive method, which would lead to the same level of global brain biodistribution. The four ventricular spaces in the brain are filled with CSF, which bathes the brain and spinal cord and protects these structures from injury. As this represents a promising route to achieve global brain coverage, several groups have attempted to exploit the ventricular system as a therapeutic approach. However, this approach displayed mixed results, often as a consequence of animal age and AAV serotype. Similarly, direct intraparenchymal administration of rAAV has also been used to achieve widespread transduction. The inclusion of the thalamic and VTA injection sites is based on the widespread projection pattern of these anatomical areas. The thalamus projects to the entire cerebral cortex, while the VTA projects to the frontal cortex and basal ganglia. Since lysosomal enzymes can be functionally transported between cells via mannose-6-phosphate receptors, these small injection sites may affect large areas of the brain.

To address these issues in the context of the MPS IIIB model, a comprehensive analysis of two capsid modified AAV8 variants compared to parental AAV8 when administered via IC6, ICV, TH, or VTA methods was performed to identify the most efficient vector and optimal administration route. A clearly superior transduction area and intensity with the IC6 AAV8 (double Y-F+T-V) was observed.

### Results

### Capsid Modification Results in Varying Effect on Brain Transduction

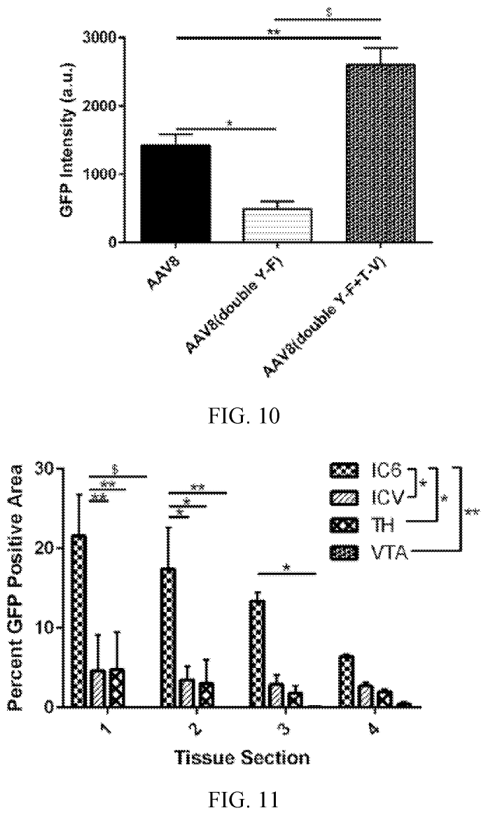

It was first sought to establish whether any differences existed between the different capsid modified AAV8 variants compared to the parental unmodified AAV8. It was previously shown that intracranial six site administration (IC6) of AAV8 leads to near global brain biodistribution within the CNS. Therefore, there was an initial assessment for changes in transduction efficiency as a consequence of capsid modification via IC6 administration. Four structural areas of functional significance within the brain were qualitatively investigated for transduction. Surprisingly, the AAV8 (double Y-F) modified vector resulted in inefficient cellular transduction compared to unmodified AAV8. However, use of AAV8 (double Y-F+T-V) resulted in robust transduction of the cortex and hippocampus, with good transduction of the thalamus and cerebellum (FIG. 9). To gauge the difference in GFP intensity as a consequence of capsid modification and mitigate the effects of GFP saturation in florescence signaling, the cortical, hippocampal, thalamic, and cerebellar regions of mid-sagittal brain sections of animals treated with each vector were analyzed using a near-infrared dye. When data were normalized to the lowest expressing vector, AAV8 (double Y-F), the use of AAV8 (double Y-F+T-V) resulted in the highest GFP intensity levels compared to unmodified AAV8 and AAV8 (double Y-F), (2612 a.u. vs. 1420 a.u. p<0.01 and 492 a.u. p<0.0001, respectively) (FIG. 10). Interestingly, it was also noted that unmodified AAV8 was superior to AAV8 (double Y-F) (p<0.05). These observations reflect a substantial difference in uptake kinetics and hints at some differences in trafficking kinetics. Taken together, it was shown that AAV8 (double Y-F+T-V)>AAV8>AAV8 (double Y-F), therefore, all subsequent experiments focused on the use of AAV8 (double Y-F+T-V).

Global Brain Biodistribution is Achieved by Modulating rAAV Delivery Route

As IC6-based vector administration represents a relatively invasive procedure, it was next sought to establish whether comparable global brain biodistribution could be similarly achieved using alternate methods, namely ICV, thalamic (TH), and VTA, compared to IC6 with AAV8 (double Y-F+T-V) delivery (FIG. 11). Each method was previously demonstrated to be a well-tolerated, effective method to achieve widespread brain biodistribution. Both ICV and TH methods represent bilateral vector administration in only two injection sites, whereas the VTA method utilizes a single injection into the parenchyma.

Three months after rAAV injection, four spatially distinct and relatively equidistant areas were selected for qualitative and quantitative assessment of GFP expression. Using the Allen Brain Atlas, the relative anatomical locations were estimated, in millimeters, medial to lateral, to be −4.2 (section 1), −3.72 (section 2), −2.72 (section 3), and −1.72 (section 4), respectively. Overall percentage area with GFP expression for all four sections was initially compared using MPS IIIB mice. Compared to the IC6 method, no other method resulted in global tissue penetration and biodistribution. Of the three other methods, ICV-delivered rAAV showed relatively confined spread to the cortical area above the hippocampus, hippocampus, and moderate spread to the thalamus and cerebellum, although lesser penetration into other tissue sections was observed. The TH delivered similar overall GFP percentage area to ICV. VTA method was far lower by comparison.

In the IC6 treated group, AAV8 (double Y-F+T-V) transduction is observed throughout the cortex, hippocampus, caudate putamen, inferior, and superior colliculus and to a lesser degree, the thalamus and cerebellum. In the cortex, robust expression was observed throughout all layers associated with somatomotor and somatosensory cues, primarily layer V. Robust expression was also observed in all layers of the frontal cortex and in orbital areas of the prefrontal cortex. Both sensory- and motor-related areas of the superior colliculus exhibited high GFP expression. In the hippocampus, widespread GFP expression was observed in all layers. In the thalamus, areas proximal to the hippocampus were transduced (FIG. 11). Furthermore, resulting vector transduction of the cerebellum was modest and resulted in GFP expression in what appeared based on morphologic assessment to be purkinje neurons, but not interneurons.

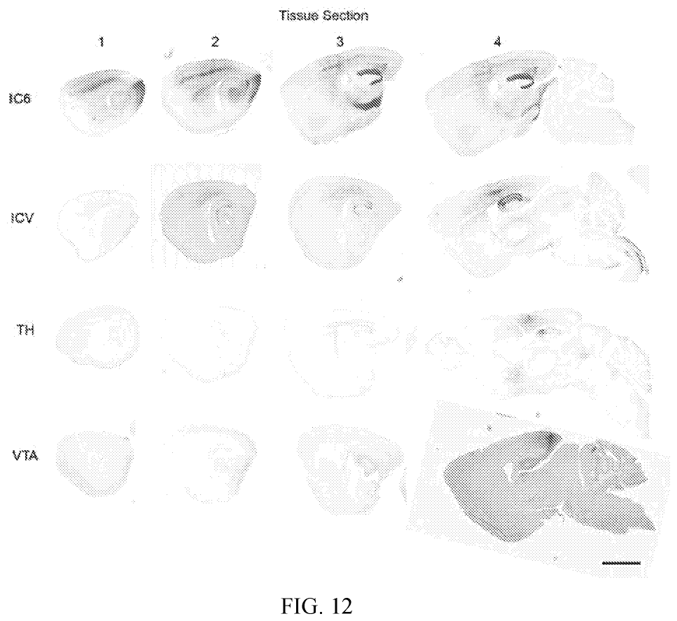

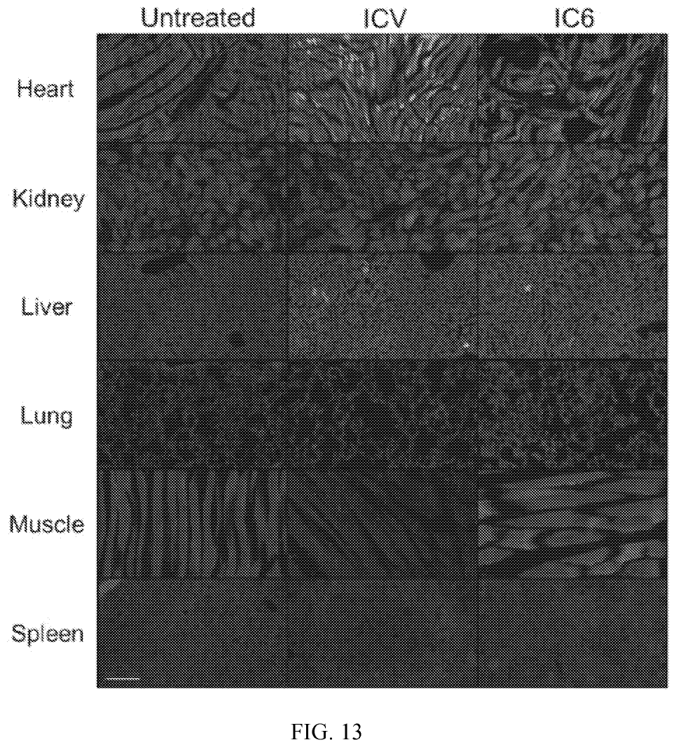

Quantitatively, diminishing but substantial GFP expression was seen in all tissue sections, overwhelmingly exhibited via the IC6 method, from ˜21% total GFP positive area in tissue section 1, down to ˜6% in section 4. It is important to note that overall, no other method of delivery achieves comparable levels of GFP biodistribution (IC6 vs. ICV and TH, p<0.05 for both, and IC6 vs. VTA, p<0.01 for all, FIG. 12). Taken together, it was concluded that IC6 rAAV administration is still the method of choice to foster global brain biodistribution.

Systemic Transduction from CNS Delivery of rAAV is Observed

As transport across the blood-brain barrier of some rAAV vectors has previously been reported, it was sought to determine whether this phenomenon would also be apparent in the MPS IIIB model. Therefore, various somatic organs were assessed including the heart, liver, muscle, kidney, and spleen of AAV8 (double Y-F+T-V) treated MPS IIIB animals. IC6 and ICV treated animals were assessed for this phenomenon, as parenchymal transport may be less likely to occur than transport out of the ventricular spaces. Interestingly, rAAV transduction into the heart and liver of treated animals was observed with both methods, but muscle transduction was not observed (FIG. 13). Compared to the IC6 method, ICV administration of AAV8 (double Y-F+T-V) resulted in a higher perceived degree of organ transduction.

### Discussion

In this Example, the impact of administration of AAV8 and capsid-mutant AAV8 vectors, AAV8 (double Y-F) and AAV8 (double Y-F+T-V) were investigated, on the efficiency of brain transduction. To maximize therapeutic potential, four brain administration methods: IC6, ICV, TH, and VTA were also investigated for their effect on modulating global biodistribution. This is the first disclosure that reveals the efficiency of capsid mutated AAV8 vectors in the CNS.

The efficiency of rAAV transduction is dependent on multiple steps involving virus-host cell interactions, which include binding to cellular receptors, overcoming intracellular barriers that limit nuclear accumulation of the virus and the conversion of single-stranded viral genomes to double-stranded forms. As previously noted, the capsid is an essential element that influences both the extracellular events related to the recognition of specific receptors and intracellular processes affecting the trafficking and uncoating. Thus, the capsid plays an essential role in the cellular tropism, transduction kinetics, and intensity of efficiency of transgene expression. Modulating these properties can improve both the effectiveness and safety of gene therapy.

Tyrosine, serine, or threonine phosphorylation serves as a signal for ubiquitination of intact AAV particles, leading to subsequent targeting for the proteasome-mediated vector degradation before reaching the nucleus. In this context, mutation of capsid tyrosine and threonine residues is predicted to allow the vectors to escape the proteasome degradation pathway and thus promote more vector genome delivery to the nucleus and more effective trans-gene expression.

The increase in transduction efficiency gained from using capsid mutated vectors has been demonstrated in several disease models. It was previously shown that IC6 administration of AAV8 leads to global biodistribution within the brain of neonatal MPS IIIB mice. To investigate the potential benefit of using capsid modified vectors, available vectors based on AAV8, namely AAV8 (double Y-F) and AAV8 (double Y-F+T-V) were selected, for comparison. The results showed a significant and robust difference in transduction efficiency among these different vectors (FIGS. 9 and 10). Although, AAV8 (double Y-F+T-V) emerged superior to AAV8 and AAV8 (double Y-F), it was surprising to find that AAV8 (double Y-F) performed worse than AAV8. Since both AAV8 (double Y-F) and AAV8 (double Y-F+T-V) are mutated at the same residues, it can be concluded that the T-V substitution in AAV8 (double Y-F+T-V) plays an important role in modulating intracellular trafficking in neural cells. In comparing the effectiveness of the method of administration in fostering global brain biodistribution, it was found that the IC6 method was far superior to the ICV, TH, and VTA methods (FIGS. 11 and 12). In subjective review of the images for the various injection methods, MPS IIIB mice appeared to have higher transduction than WT with the both the AAV8 (double Y-F+T-V) and with native AAV8, in agreement with prior publications also showing higher transduction with AAV8 in MPS IIIB than WT.

The possibility that some of this effect may be related to differing total volume of injection cannot be ruled out. The maximum injection volume for the Hamilton syringe is 4 μl and it was uncomfortable injecting more than this in any one site due to concerns for displacement of tissue and inducing further artifact by repeated needle placement at a site in order to reload the syringe and increase volume. Therefore, the concentrations of vector were adjusted to fit the same total vector copy number/injection method in the volume that could comfortably be injected with one syringe load for each site per method.

Given the unique distribution profiles associated with each method, a disease-specific method of administration should be considered. For example, the ICV method resulted in very high transduction of the hippocampus, and parts of the cortex and thalamus (FIG. 11). This method of administration may be more effective in the treatment of Alzheimer's, where severe pathology first affects parts of the cortex and the hippocampus. Similarly, a single VTA injection resulted in localized deposition in the mid-brain. This application may be better suited to the treatment of Parkinson's disease, which affects cells in a localized region, the substantia nigra. Furthermore, consistent with previous findings, brain to systemic spread of AAV8 (double Y-F+T-V) was also demonstrated, which was more pronounced when administered via ICV delivery (FIG. 13).

In conclusion, the present disclosure is the first to analyze the transduction capacity of AAV8 capsid tyrosine/threonine modified vectors and their impact on brain transduction in the MPS IIIB model. As successful treatment of MPS IIIB will require global vector biodistribution and tissue penetration, it was also sought to identify an optimal method of delivery which would maximize therapeutic potential. Taken together, the data revealed that IC6 administration of an AAV8 (double Y-F+T-V) vector results in enhanced biodistribution of transgene expression in the CNS. It is expected that when combined with a codon optimized α-N-acetylglucosaminidase (NAGLU) this will result in increased therapeutic benefit, although the modest transport of vector into systemic circulation may warrant use of immunomodulatory agents.

### Materials and Methods

### Mice

The congenic C57BL/6 NAGLU-deficient mouse strain was maintained and expanded by strict sibling mating. Wild type (+/+), heterozygous (±) (subsequently referred to as “Control”) and mutant (−/−). Genotyping was done on tissue of newborn mice (P2-3) by enzyme assay or NAGLU exon 6 and neomycin insertion cassette PCR.

### AAV Constructs

Recombinant AAV2 plasmids pseudotyped with capsid proteins from AAV8 were produced, purified, and titered. Vector titer was determined by dot blot assays, diluted, aliquoted, and stored at −80° C. until use.

Site-directed mutagenesis of surface-exposed tyrosine residues on AAV2 capsids has been described recently. Similar strategies were used to generate AAV serotype 8 vectors containing tyrosine to phenylalanine mutations. Vector preparations were produced using the plasmid co-transfection method.

Each of the three single-stranded rAAV vectors, AAV8, AAV8 (double Y-F), and AAV8 (double Y-F+T-V), express humanized green fluorescent protein driven by the hybrid cytomegalovirus enhancer/chicken beta-actin promoter.

### Treatments

All treatments were performed in genotyped pups at 3-4 days of age and were well tolerated. For each comparison, 3-5 control and 3-5 MPS IIIB neonatal mice were injected by one of four methods; intracranial six site (IC6, n=5), ICV (n=3), thalamic (TH, n=3), or VTA (n=3). Neonates were cryoanesthetized prior to and during treatment and were then placed on a warming pad after treatment, before being returned to their mothers. All treatments were well tolerated. Intracranial rAAV-GFP was administered using the following coordinates determined by ruler: IC6-bilateral frontal (from bregma: 2 mm lateral, 1 mm posterior, and 1.5 mm deep), bilateral temporal (from bregma: 3 mm lateral, 3 mm posterior, and 2.5 mm deep), and bilateral cerebellar (from lambda: 1 mm lateral and posterior, 1.5 mm deep); TH (bilateral injections from bregma: 4 mm lateral, 1 mm posterior, and 3 mm deep); ICV-needle was placed perpendicular to the skull surface bilaterally (0.25 mm lateral to the sagittal suture, 0.50-0.75 mm rostral to the neonatal coronary suture, and 2 mm deep); and VTA (unilateral injection from bregma: 4.2 mm lateral and 4 mm deep; modified from Wolfe et al.). All injections were conducted by hand through the skull using a 32-gauge Hamilton syringe (Narishige Int., East Meadow, N.Y.). All mice received a total of 1.4×10¹⁰ vector genomes in 4-12 μl volumes over 1-3 minutes.

### Histological Procedures

Animals were sacrificed 3 months after vector infusion. Mice were euthanized with 100 μl of Ketamine (120 mg/kg)/Xylazine (16 mg/kg) cocktail followed by thoracotomy. Transcardial perfusion with 1xPBS followed by fresh ice-cold 4% paraformaldehyde in 1xPBS solution followed. Brains were harvested, post fixed for 3 hours in 4% paraformaldehyde at 4° C., followed by overnight incubation in 20% sucrose in 1xPBS at 4° C. One brain hemisphere was then embedded in OCT (Triangle Biomedical Sciences, Durham, N.C.) and rapidly frozen in a 2-methyl-butane/dry ice bath. Sagittal sections were cut to a thickness of 20 μm and stored in a cryoprotective solution at −80° C. until use.

### Quantitation of GFP

Quantitation of GFP positive area was conducted using the Scanscope FL instrument (Aperio Technologies, Vista, Calif.). Analysis was conducted using accompanying Image-Scope software and the Positive Pixel Count FL v1 algorithm. Use of the tuning feature allowed for maximal capture of GFP based on pixel intensity. The minimum intensity was set between 0.2 and 0.22, and maximum intensity was set to 1. Regions of interest were demarcated using the Allen Reference Atlas as a neuroanatomical reference. GFP positive area was determined using the average from three independent tests performed by three readers in a blinded manner.

To reliably assess differences in GFP intensity as a consequence of AAV capsid modification, the Odyssey Infrared Imaging system (Li-Cor, Lincoln, Nebr.) was utilized. Briefly, mid-sagittal brain sections of AAV8, AAV8 (double Y-F), or AAV8 (double Y-F+T-V) vector treated animals were incubated overnight with GFP anti-body (1:2000, Abcam, Cambridge, Mass.; Cat. #: ab290) in 1xPBS/0.01% TBS-T/10% NDS/1% BSA buffer. To visualize GFP, donkey anti-rabbit 680 DR was used (1:5000, Li-Cor Biosciences, Lincoln, Nebr., Cat. #926-68073). Sections are then mounted to slides and allowed to dry overnight followed by clearing with Xylene. Slide mounted sections were again air-dried overnight and scanned using the Odyssey system the following day. Analysis was conducted using the Image Studio Lite version 4.0 software (Li-Cor Biosciences, Lincoln, Nebr.).

### Statistical Analysis

Sample sizes for groups were based on previously observed effect size in intensity of 4 and GFP percent area of 3 between AAV8 in control and MPS IIIB. In order to identify an effect size of 3.14 with alpha error of 0.05 and power of 0.8, a group size of three is required. GraphPad Prism 6 was used for statistical analysis. Two-tailed Stu-dent's t test was used for unpaired data. Brown-Forsythe test was performed to confirm equal variance and Tukey's test was used to correct for multiple comparisons for ANOVA analysis. For comparisons with unequal variance, a log transform was used to normalize the data and reduce heterogeneity. Bar graphs are shown as mean±SEM. Probability p<0.05 was considered statistically significant. Animals were allocated to injection method by sequential selection of alternating method for each virus of animals randomly selected from each genotype. Allocation of areas of analysis for histology was carried out in a blinded fashion but the sample preparation and analysis itself was not blinded. From the intention-to-treat animals, those with evidence of vector extrusion during injection were excluded from analysis.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be non-limiting and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A recombinant adeno-associated viral (rAAV) vector comprising a heterologous nucleic acid encoding N-acetylglucosaminidase alpha (NAGLU), wherein the heterologous nucleic acid comprises a sequence having at least 85% identity, at least 90% identity, at least 92.5% identity, at least 95% identity, at least 98% identity, or at least 99% identity to the sequence of SEQ ID NO: 1, and wherein the rAAV vector is of serotype AAV8, or a variant thereof.

2. The rAAV vector of claim 1, wherein the heterologous nucleic acid comprises a nucleic acid sequence that comprises the sequence set forth as SEQ ID NO: 1.

3. The rAAV vector of claim 1 or 2, wherein the rAAV vector is of serotype variant AAV8(Y447F+Y733F+T494V).

4. The rAAV vector of any one of claims 1-3, wherein the vector comprises a heterologous nucleic acid sequence comprising a sequence having at least 80% identity, at least 85% identity, at least 90% identity, at least 92.5% identity, at least 95% identity, at least 98% identity, or at least 99% identity to the sequence of SEQ ID NO: 2.

5. The rAAV vector of any one of claims 1-4, wherein the vector comprises a nucleic acid sequence that comprises the sequence set forth as SEQ ID NO: 2.

6. The rAAV vector of any one of claims 1-5, wherein the vector comprises a chicken beta-actin (CBA) promoter that is operably linked to the heterologous nucleic acid.

7. The rAAV vector of any one of claims 1-6, wherein the vector comprises inverted terminal repeats from AAV serotype 2 (AAV2).

8. The rAAV vector of any one of claims 1-7, wherein the heterologous nucleic acid is codon-optimized for human expression.

9. An rAAV particle comprising the rAAV vector of any one of claims 1-8.

10. The rAAV particle of claim 9 further comprising a capsid variant of the AAV8(Y447F+Y733F+T494V) or the AAV8(Y447F+Y733F) serotype.

11. A pharmaceutical comprising the rAAV particle of claim 9 or 10, and one or more pharmaceutically acceptable excipients.

12. A method of treatment of a subject having, or at risk of having, Sanfilippo syndrome, by administering to the subject the rAAV particle of claim 9 or 10, or the pharmaceutical composition of claim 11.

13. The method of claim 12, wherein the rAAV particle or the pharmaceutical composition is administered by intraparenchymal injection or cisternal injection.

14. The method of claim 12 or 13, wherein the rAAV particle or the pharmaceutical composition is administered by intraparenchymal six site (IC6) injection.

15. The method of claim 12, wherein the rAAV particle or the pharmaceutical composition is administered by intracerebroventricular (ICV) injection, intrathalamic injection, or ventral tegmental area (VTA) injection.

16. The method of any one of claims 12-15, wherein the administration results in partial or complete restoration of hearing loss in the subject.

17. The method of any one of claims 12-16, wherein the administration results in partial or complete restoration of normal levels of heparan sulfate storage in the subject.

18. The method of any one of claims 12-17, wherein the subject is a human.

19. A method of transducing expression of NAGLU in one or more brain cells comprising administering to the one or more cells the rAAV vector of any one of claims 1-7.

20. The method of claim 19, wherein the one or more brain cells comprise cells of the cerebral cortex, hippocampus, thalamus or cerebellum.

21. The method of claim 19 or 20, wherein the one or more brain cells comprise cells of the cerebral cortex.

22. A host cell comprising the rAAV vector of any one of claims 1-8.

23. The host cell of claim 22, wherein the host cell is a human neuron or glia.

24. A recombinant adeno-associated viral (rAAV) vector comprising a heterologous nucleic acid encoding NAGLU, wherein the heterologous nucleic acid comprises a sequence having at least 85% identity, at least 90% identity, at least 92.5% identity, at least 95% identity, at least 98% identity, or at least 99% identity to the sequence of SEQ ID NO: 7, and wherein the rAAV vector is of serotype AAV8, or a variant thereof.

25. The rAAV vector of claim 24, wherein the heterologous nucleic acid comprises a nucleic acid sequence that comprises the sequence set forth as SEQ ID NO: 7.

26. An rAAV particle comprising the rAAV vector of claim 24 or 25.

27. A method of treatment of a subject having, or at risk of having, Sanfilippo syndrome, by administering to the subject the rAAV particle of claim 26.
